(19) United States
(12) Patent Application Publication
(10) Pub. No.: US 2024/0417453 A1
(43) Pub. Date: Dec. 19, 2024

(54) IL-2 FUSION PROTEINS, PHARMACEUTICAL COMPOSITIONS, AND THERAPEUTIC APPLICATIONS

(71) Applicant: Anwita Biosciences, Inc., San Carlos, CA (US)

(72) Inventors: Ziyang Zhong, Belmont, CA (US); Fan Ye, Mountain View, CA (US); Jianing Huang, San Mateo, CA (US)

(21) Appl. No.: 18/703,976

(22) PCT Filed: Oct. 26, 2022
(86) PCT No.: PCT/US2022/078690

§ 371 Date: Apr. 23, 2024

Related U.S. Application Data
(60) Provisional application No. 63/263,085, filed on Oct. 27, 2021.

(51) Int. Cl. C07K 16/18; A61K 38/00; A61P 35/00; C07K 14/55

(52) U.S. Cl. CPC C07K 16/18; A61P 35/00; C07K 14/55; A61K 38/00; C07K 2317/24; C07K 2317/53; C07K 2317/569; C07K 2317/92; C07K 2319/00

(57) ABSTRACT

Provided herein are an IL-2 fusion protein and a pharmaceutical composition thereof. Also provided herein are methods of their use for treating, preventing, or ameliorating one or more symptoms of a proliferative disease.

### CROSS REFERENCE TO RELATED APPLICATION

This application claims the benefit of the priority of U.S. Provisional Application No. 63/263,085, filed Oct. 27, 2021; the disclosure of which is incorporated herein by reference in its entirety.

### FIELD

Provided herein are an IL-2 fusion protein and a pharmaceutical composition thereof. Also provided herein are methods of their use for treating, preventing, or ameliorating one or more symptoms of a proliferative disease.

### REFERENCE TO A SEQUENCE LISTING

The present specification is being filed with a Sequence Listing entitled 216A016WO01_SEQLIST_ST26.XML of 112,159 bytes in size and created Oct. 26, 2022; the content of which is incorporated herein by reference in its entirety.

### BACKGROUND

An interleukin-2 (IL-2) is a pleiotropic cytokine that orchestrates the proliferation, survival, and function of both immune effector (Teff) cells and regulatory T (Treg) cells to maintain immune homeostasis. Bluestone, N. Engl. J. Med. 2011, 365, 2129-31; Boyman et al., Nat. Rev. Immunol. 2012, 12, 180-90. The IL-2 drives T-cell growth, augments natural killer (NK) cytolytic activity, induces the differentiation of regulatory T (Treg) cells, and mediates activation-induced cell death. Liao et al., Curr. Opin. Immunol. 2011, 23, 598-604.

An IL-2 receptor (IL-2R) has three different IL-2 receptor chains: α chain (IL-2Rα or CD25), β chain (IL-2Rβ or CD122), and 7 chain (IL-2Rγ, γ_{c}, or CD132). Wang et al., Science 2005, 310, 1159-63. The IL-2 binds the IL-2Rα with a low affinity (K_{d}≈10 nM). Id. From a crystal structure of a quaternary IL-2 signaling complex, fifteen amino acid residues (K35, T37, R38, T41, F42, K43, F44, Y45, E61, E62, K64, P65, E68, L72, and Y107) on the IL-2 are identified as interface residues between the IL-2 and IL-2Rα. Stauber et al., Proc. Natl. Acad. Sci. U.S.A. 2006, 103, 2788-93. The IL-2 binds a heterodimeric complex of the IL-2Rβ and IL-2Rγ (“IL-2Rβ/γ”), expressed on memory T cells and NK cells, with an intermediate affinity (K_{d}≈1 nM). Wang et al., Science 2005, 310, 1159-63. The IL-2 binds a heterotrimeric complex of the IL-2Rα, IL-2Rβ, and IL-2Rγ, expressed on Treg cells, with a high affinity (K_{d}≈10 pM). Id. The TL-2 binds the IL-2Rβ alone with a dissociation constant (K_{d}) of about 100 nM and has no detectable binding affinity to the IL-2Rγ alone. Id. The IL-2Rα by itself has no signal-transducing activity. Id. The IL-2 signals through the intermediate-affinity heterodimeric IL-2Rβ/γ complex or the high-affinity heterotrimeric IL-2Rα/β/γ complex. Liao et al., Curr. Opin. Immunol. 2011, 23, 598-604. The binding of the TL-2 to the intermediate-affinity heterodimeric IL-2Rβ/γ complex leads to the activation and proliferation of immunostimulatory Teff cells, while the binding of the IL-2 to the high-affinity heterotrimeric IL-2Rα/β/γ complex results in the activation and proliferation of immunosuppressive Treg cells. Malek et al., Immunity 2010, 33, 153-65; Bluestone, N. Engl. J Med. 2011, 365, 2129-31; Boyman et al., Nat. Rev. Immunol. 2012, 12, 180-90; Spangler et al., Annu. Rev. Immunol. 2015, 33, 139-67. These dual opposing functions of immunostimulation and immunosuppression pose a major challenge in developing the TL-2 as a safe and effective therapeutic agent. Skrombolas et al., Expert Rev. Clin. Immunol. 2014, 10, 207-17; Abbas et al., Sci. Immunol. 2018, 3, eaat 1482.

Aldesleukin, a recombinant human IL-2, was approved by the FDA in 1992 for metastatic renal cell carcinoma and in 1998 for metastatic melanoma. Rosenberg, J. Immunol. 2014, 192, 5451-58. Patients with metastatic melanoma or renal cancer experience a 5 to 10% rate of complete cancer regression, with an additional 10% experiencing a partial regression. Atkins et al., J. Clin. Oncol. 1999, 17, 2105-16; Klapper et al., Cancer 2008, 113, 293-301. Approximately 70% of complete responders to the IL-2 therapy do not recur. Rosenberg, Sci. Transl. Med. 2012, 4, 127ps8. However, the success of the TL-2 as an immunotherapy for cancer has been hampered by its severe toxicities and limited efficacy. One major limiting factor for its efficacy as an anticancer agent is immunosuppression resulting from the IL-2-driven preferential expansion of Treg cells. Abbas et al., Sci. Immunol. 2018, 3, eaat 1482. A high dose therapeutic schedule is required for the IL-2 to be effective in cancer treatment. Bluestone, N. Engl. J. Med. 2011, 365, 2129-31; Abbas et al., Sci. Immunol. 2018, 3, eaat 1482. This dosing regimen, however, causes vascular leak syndrome and limits the application of IL-2 in cancer treatment. Abbas et al., Sci. Immunol. 2018, 3, eaat 1482. Therefore, there is a need for an effective IL-2 immunotherapy for treating cancer.

### SUMMARY OF THE DISCLOSURE

Provided herein is a fusion protein comprising an interleukin-2 (IL-2) domain, a single domain antibody (sdAb) that binds to a human serum albumin (HSA) (i.e., anti-HSA sdAb), and optionally a peptide linker; wherein the carboxyl terminus (C-terminus) of the anti-HSA sdAb is connected to the amino terminus (N-terminus) of the IL-2 domain directly or via the peptide linker.

Also provided herein is a fusion protein comprising an IL-2 domain, an anti-HSA sdAb, and optionally a peptide linker; wherein the C-terminus of the anti-HSA sdAb is connected to the N-terminus of the IL-2 domain directly or via the peptide linker; and wherein the IL-2 domain of SEQ ID NO: 1 comprises (i) a replacement of the amino acid residues from positions 29 to 50 as set forth in SEQ ID NO: 1 with a peptide comprising the amino acid sequence of an IL-15 hinge or a fragment thereof and (ii) one or more amino acid substitutions.

Additionally provided herein is a pharmaceutical composition comprising a fusion protein provided herein; and a pharmaceutically acceptable excipient.

Furthermore, provided herein is a method of treating, preventing, or ameliorating one or more symptoms of a proliferative disease in a subject, comprising administering to the subject a therapeutically effective amount of a fusion protein provided herein.

Provided herein is a method of activating an immune effector cell, comprising contacting the cell with an effective amount of a fusion protein provided herein.

### BRIEF DESCRIPTION OF THE DRAWINGS

FIG. 1 shows a sensorgraph of the interaction of anti-HSA-hIL-2 fusion protein C1 with the human IL-2Rα, where the fusion protein was tested at 33.3 nM, 100 nM, and 300 nM.

FIG. 2 shows a sensorgraph of the interaction of anti-HSA-hIL-2 fusion protein A2 with the human IL-2Rα, where the fusion protein was tested at 33.3 nM, 100 nM, and 300 nM.

FIG. 3 shows a sensorgraph of the interaction of anti-HSA-hIL-2 fusion protein A4 with the human IL-2Rα, where the fusion protein was tested at 33.3 nM, 100 nM, and 300 nM.

FIG. 4 shows a sensorgraph of the interaction of anti-HSA-hIL-2 fusion protein C1 with the human IL-2Rβ, where the fusion protein was tested at 200 nM, 400 nM, and 800 nM.

FIG. 5 shows a sensorgraph of the interaction of anti-HSA-hIL-2 fusion protein A2 with the human IL-2Rβ, where the fusion protein was tested at 200 nM, 400 nM, and 800 nM.

FIG. 6 shows a sensorgraph of the interaction of anti-HSA-hIL-2 fusion protein A4 with the human IL-2Rβ, where the fusion protein was tested at 200 nM, 400 nM, and 800 nM.

### DETAILED DESCRIPTION

To facilitate understanding of the disclosure set forth herein, a number of terms are defined below.

Generally, the nomenclature used herein and the laboratory procedures in biochemistry, biology, cell biology, molecular biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of its attendant symptoms; or alleviating or eradicating the cause(s) of the disorder, disease, or condition itself.

The terms “prevent,” “preventing,” and “prevention” are meant to include delaying and/or precluding the onset of a disorder, disease, or condition, and/or one or more of its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “substantially pure” and “substantially homogeneous” mean sufficiently homogeneous to appear free of readily detectable impurities as determined by a standard analytical method used by one of ordinary skill in the art, including, but not limited to, gel electrophoresis, high performance liquid chromatography (HPLC), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 80%, at least about 90%, at least about 92%, at least about 95%, at least about 96%, at least about 97%, at least about 98%, at least about 99%, or at least about 99.5% by weight of the molecules are a single compound as determined by a standard analytical method.

### IL-2 Fusion Proteins

In one embodiment, provided herein is a fusion protein comprising an interleukin-2 (IL-2) domain, a single domain antibody (sdAb) that binds to a human serum albumin (HSA) (i.e., anti-HSA sdAb), and optionally a peptide linker; wherein the carboxyl terminus (C-terminus) of the anti-HSA sdAb is connected to the amino terminus (N-terminus) of the IL-2 domain directly or via the peptide linker.

In one embodiment, the IL-2 domain in a fusion protein provided herein comprises an amino acid sequence of SEQ ID NO: 1.

In another embodiment, provided herein is a fusion protein comprising an IL-2 domain, an anti-HSA sdAb, and optionally a peptide linker; wherein the C-terminus of the anti-HSA sdAb is connected to the N-terminus of the IL-2 domain directly or via the peptide linker; and wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof (“an IL-15 hinge fragment-containing peptide”) and (ii) an amino acid substitution (i.e., one or more amino acid substitutions).

In yet another embodiment, provided herein is a fusion protein comprising an IL-2 domain, an anti-HSA sdAb, and optionally a peptide linker; wherein the C-terminus of the anti-HSA sdAb is connected to the N-terminus of the IL-2 domain directly or via the peptide linker; and wherein the IL-2 domain comprises (i) a replacement of the amino acid residues from positions 29 to 50 as set forth in SEQ ID NO: 1 with an IL-15 hinge fragment-containing peptide, and (ii) an amino acid substitution at position 3, 8, 9, 13, 15, 16, 18, 19, 23, 32, 76, 79, 81, 84, 87, 88, 91, 92, 95, 125, 126, 127, or 130 as set forth in SEQ ID NO: 1.

In one embodiment, the IL-2 domain in the fusion protein provided herein comprises (i) a replacement of the amino acid residues from positions N29 to A50 as set forth in SEQ ID NO: 1 with an IL-15 hinge fragment-containing peptide; and (ii) an amino acid substitution at position T3, K8, K9, Q13, E15, H16, L18, L19, M23, K32, K76, H79, R81, D84, S87, N88, V91, I92, E95, C125, Q126, S127, or S130 as set forth in SEQ ID NO: 1.

In certain embodiments, the amino acid at position 3 as set forth in SEQ ID NO: 1 is one of the twenty natural amino acids (i.e., Ala (A), Cys (C), Asp (D), Glu (E), Phe (F), Gly (G), His (H), Ile (I), Lys (K), Leu (L), Met (M), Asn (N), Pro (P), Gln (Q), Arg (R), Ser (S), Thr (T), Val (V), Trp (W), and Tyr (Y)) other than T. In certain embodiments, the amino acid at position 3 as set forth in SEQ ID NO: 1 is A.

In certain embodiments, the amino acid at position 8 as set forth in SEQ ID NO: 1 is one of the twenty natural amino acids other than K. In certain embodiments, the amino acid at position 8 as set forth in SEQ ID NO: 1 is D, E, or Q. In certain embodiments, the amino acid at position 8 as set forth in SEQ ID NO: 1 is D. In certain embodiments, the amino acid at position 8 as set forth in SEQ ID NO: 1 is E. In certain embodiments, the amino acid at position 8 as set forth in SEQ ID NO: 1 is Q.

In certain embodiments, the amino acid at position 9 as set forth in SEQ ID NO: 1 is one of the twenty natural amino acids other than K. In certain embodiments, the amino acid at position 9 as set forth in SEQ ID NO: 1 is D, E, or Q. In certain embodiments, the amino acid at position 9 as set forth in SEQ ID NO: 1 is D. In certain embodiments, the amino acid at position 9 as set forth in SEQ ID NO: 1 is E. In certain embodiments, the amino acid at position 9 as set forth in SEQ ID NO: 1 is Q.

In certain embodiments, the amino acid at position 13 as set forth in SEQ ID NO: 1 is one of the twenty natural amino acids other than Q. In certain embodiments, the amino acid at position 13 as set forth in SEQ ID NO: 1 is E or N. In certain embodiments, the amino acid at position 13 as set forth in SEQ ID NO: 1 is E. In certain embodiments, the amino acid at position 13 as set forth in SEQ ID NO: 1 is N.

In certain embodiments, the amino acid at position 15 as set forth in SEQ ID NO: 1 is one of the twenty natural amino acids other than E. In certain embodiments, the amino acid at position 15 as set forth in SEQ ID NO: 1 is K, Q, or V. In certain embodiments, the amino acid at position 15 as set forth in SEQ ID NO: 1 is K or Q. In certain embodiments, the amino acid at position 15 as set forth in SEQ ID NO: 1 is K. In certain embodiments, the amino acid at position 15 as set forth in SEQ ID NO: 1 is Q. In certain embodiments, the amino acid at position 15 as set forth in SEQ ID NO: 1 is V.

In certain embodiments, the amino acid at position 16 as set forth in SEQ ID NO: 1 is one of the twenty natural amino acids other than H. In certain embodiments, the amino acid at position 16 as set forth in SEQ ID NO: 1 is D, E, N, or Q. In certain embodiments, the amino acid at position 16 as set forth in SEQ ID NO: 1 is D, E, or Q. In certain embodiments, the amino acid at position 16 as set forth in SEQ ID NO: 1 is D. In certain embodiments, the amino acid at position 16 as set forth in SEQ ID NO: 1 is E. In certain embodiments, the amino acid at position 16 as set forth in SEQ ID NO: 1 is N. In certain embodiments, the amino acid at position 16 as set forth in SEQ ID NO: 1 is Q.

In certain embodiments, the amino acid at position 18 as set forth in SEQ ID NO: 1 is one of the twenty natural amino acids other than L. In certain embodiments, the amino acid at position 18 as set forth in SEQ ID NO: 1 is C.

In certain embodiments, the amino acid at position 19 as set forth in SEQ ID NO: 1 is one of the twenty natural amino acids other than L. In certain embodiments, the amino acid at position 19 as set forth in SEQ ID NO: 1 is S.

In certain embodiments, the amino acid at position 23 as set forth in SEQ ID NO: 1 is one of the twenty natural amino acids other than M. In certain embodiments, the amino acid at position 23 as set forth in SEQ ID NO: 1 is K.

In certain embodiments, the amino acid at position 32 as set forth in SEQ ID NO: 1 is one of the twenty natural amino acids other than K. In certain embodiments, the amino acid at position 32 as set forth in SEQ ID NO: 1 is D, E, H, or Q. In certain embodiments, the amino acid at position 32 as set forth in SEQ ID NO: 1 is D. In certain embodiments, the amino acid at position 32 as set forth in SEQ ID NO: 1 is E. In certain embodiments, the amino acid at position 32 as set forth in SEQ ID NO: 1 is H. In certain embodiments, the amino acid at position 32 as set forth in SEQ ID NO: 1 is Q.

In certain embodiments, the amino acid at position 76 as set forth in SEQ ID NO: 1 is one of the twenty natural amino acids other than K. In certain embodiments, the amino acid at position 76 as set forth in SEQ ID NO: 1 is D, E, or Q. In certain embodiments, the amino acid at position 76 as set forth in SEQ ID NO: 1 is D. In certain embodiments, the amino acid at position 76 as set forth in SEQ ID NO: 1 is E. In certain embodiments, the amino acid at position 76 as set forth in SEQ ID NO: 1 is Q.

In certain embodiments, the amino acid at position 79 as set forth in SEQ ID NO: 1 is one of the twenty natural amino acids other than H. In certain embodiments, the amino acid at position 79 as set forth in SEQ ID NO: 1 is D, E, or Q. In certain embodiments, the amino acid at position 79 as set forth in SEQ ID NO: 1 is D. In certain embodiments, the amino acid at position 79 as set forth in SEQ ID NO: 1 is E. In certain embodiments, the amino acid at position 79 as set forth in SEQ ID NO: 1 is Q.

In certain embodiments, the amino acid at position 81 as set forth in SEQ ID NO: 1 is one of the twenty natural amino acids other than R. In certain embodiments, the amino acid at position 81 as set forth in SEQ ID NO: 1 is D, E, or Q. In certain embodiments, the amino acid at position 81 as set forth in SEQ ID NO: 1 is D. In certain embodiments, the amino acid at position 81 as set forth in SEQ ID NO: 1 is E. In certain embodiments, the amino acid at position 81 as set forth in SEQ ID NO: 1 is Q.

In certain embodiments, the amino acid at position 84 as set forth in SEQ ID NO: 1 is one of the twenty natural amino acids other than D. In certain embodiments, the amino acid at position 84 as set forth in SEQ ID NO: 1 is T.

In certain embodiments, the amino acid at position 87 as set forth in SEQ ID NO: 1 is one of the twenty natural amino acids other than S. In certain embodiments, the amino acid at position 87 as set forth in SEQ ID NO: 1 is D or E. In certain embodiments, the amino acid at position 87 as set forth in SEQ ID NO: 1 is D. In certain embodiments, the amino acid at position 87 as set forth in SEQ ID NO: 1 is E.

In certain embodiments, the amino acid at position 88 as set forth in SEQ ID NO: 1 is one of the twenty natural amino acids other than N. In certain embodiments, the amino acid at position 88 as set forth in SEQ ID NO: 1 is A or R. In certain embodiments, the amino acid at position 88 as set forth in SEQ ID NO: 1 is A. In certain embodiments, the amino acid at position 88 as set forth in SEQ ID NO: 1 is R.

In certain embodiments, the amino acid at position 91 as set forth in SEQ ID NO: 1 is one of the twenty natural amino acids other than V. In certain embodiments, the amino acid at position 91 as set forth in SEQ ID NO: 1 is I.

In certain embodiments, the amino acid at position 92 as set forth in SEQ ID NO: 1 is one of the twenty natural amino acids other than I. In certain embodiments, the amino acid at position 92 as set forth in SEQ ID NO: 1 is A or L. In certain embodiments, the amino acid at position 92 as set forth in SEQ ID NO: 1 is A. In certain embodiments, the amino acid at position 92 as set forth in SEQ ID NO: 1 is L.

In certain embodiments, the amino acid at position 95 as set forth in SEQ ID NO: 1 is one of the twenty natural amino acids other than E. In certain embodiments, the amino acid at position 95 as set forth in SEQ ID NO: 1 is K, N, or Q. In certain embodiments, the amino acid at position 95 as set forth in SEQ ID NO: 1 is K or Q. In certain embodiments, the amino acid at position 95 as set forth in SEQ ID NO: 1 is K. In certain embodiments, the amino acid at position 95 as set forth in SEQ ID NO: 1 is N. In certain embodiments, the amino acid at position 95 as set forth in SEQ ID NO: 1 is Q.

In certain embodiments, the amino acid at position 125 as set forth in SEQ ID NO: 1 is one of the twenty natural amino acids other than C. In certain embodiments, the amino acid at position 125 as set forth in SEQ ID NO: 1 is A or S. In certain embodiments, the amino acid at position 125 as set forth in SEQ ID NO: 1 is A. In certain embodiments, the amino acid at position 125 as set forth in SEQ ID NO: 1 is S.

In certain embodiments, the amino acid at position 126 as set forth in SEQ ID NO: 1 is one of the twenty natural amino acids other than Q. In certain embodiments, the amino acid at position 126 as set forth in SEQ ID NO: 1 is E.

In certain embodiments, the amino acid at position 127 as set forth in SEQ ID NO: 1 is one of the twenty natural amino acids other than S. In certain embodiments, the amino acid at position 127 as set forth in SEQ ID NO: 1 is A, E, F, or W. In certain embodiments, the amino acid at position 127 as set forth in SEQ ID NO: 1 is A. In certain embodiments, the amino acid at position 127 as set forth in SEQ ID NO: 1 is E. In certain embodiments, the amino acid at position 127 as set forth in SEQ ID NO: 1 is F. In certain embodiments, the amino acid at position 127 as set forth in SEQ ID NO: 1 is W.

In certain embodiments, the amino acid at position 130 as set forth in SEQ ID NO: 1 is one of the twenty natural amino acids other than S. In certain embodiments, the amino acid at position 130 as set forth in SEQ ID NO: 1 is A, E, F, or W. In certain embodiments, the amino acid at position 130 as set forth in SEQ ID NO: 1 is A. In certain embodiments, the amino acid at position 130 as set forth in SEQ ID NO: 1 is E. In certain embodiments, the amino acid at position 130 as set forth in SEQ ID NO: 1 is F. In certain embodiments, the amino acid at position 130 as set forth in SEQ ID NO: 1 is W.

In one embodiment, the IL-2 domain in the fusion protein provided herein comprises (i) a replacement of the amino acid residues from positions N29 to A50 of SEQ ID NO: 62 with an IL-15 hinge fragment-containing peptide; and (ii) an amino acid substitution of T3A, K8D, K8E, K8Q, K9D, K9E, K9Q, Q13E, Q13N, E15K, E15Q, E15V, H16D, H16E, H16N, H16Q, L18C, L19S, M23K, K32D, K32E, K32Q, K76D, K76E, K76Q, H79D, H79E, H79Q, R81D, R81E, R81Q, D84T, S87D, S87E, N88A, V91I, I92A, I92L, E95K, E95N, E95Q, C125A, C125S, Q126E, S127A, S127E, S127F, S127W, S130A, S130E, S130F, or S130W.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position 3, 8, 9, 15, 16, 18, 32, 76, 79, 81, 84, 87, 88, 92, 95, 125, 126, 127, or 130 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position T3, K8, K9, E15, H16, L18, K32, K76, H79, R81, D84, S87, N88, 192, E95, C125, Q126, S127, or S130 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of T3A, K8D, K8E, K8Q, K9D, K9E, K9Q, E15K, E15Q, H16D, H16E, H16Q, L18C, K32D, K32E, K32Q, K76D, K76E, K76Q, H79D, H79E, H79Q, R81D, R81E, R81Q, D84T, S87D, S87E, N88A, I92A, E95K, E95Q, C125A, C125S, Q126E, S127A, S127E, S127F, S127W, S130A, S130E, S130F, or S130W.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position 3, 15, 18, 32, 76, 87, 125, or 126 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position T3, E15, L18, K32, K76, S87, C125, or Q126 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of T3A, E15K, E15Q, L18C, K32D, K32E, K32Q, K76D, K76E, K76Q, S87D, S87E, C125A, C125S, or Q126E.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position 3, 15, 18, 87, 125, or 126 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position T3, E15, L18, S87, C125, or Q126 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of T3A, E15K, E15Q, L18C, S87D, S87E, C125A, C125S, or Q126E.

In still another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of T3A, E15K, L18C, S87D, S87E, C125S, or Q126E.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position 3, 15, 87, or 125 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position T3, E15, S87, or C125 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of T3A, E15K, E15Q, S87D, S87E, C125A, or C125S.

In still another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of T3A, E15K, S87D, S87E, or C125S.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions, each independently at position 3, 15, 87, or 125 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions, each independently at position T3, E15, S87, or C125 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions, each independently selected from T3A, E15K, E15Q, S87D, S87E, C125A, and C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions of E15K, and S87D or S87E.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions of E15K and C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions of S87D or S87E, and C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions of S87D and C125S.

In still another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) two amino acid substitutions of S87E and C125S.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) three amino acid substitutions, each independently at position 3, 15, 87, or 125 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) three amino acid substitutions, each independently at position T3, E15, S87, or C125 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) three amino acid substitutions, each independently selected from T3A, E15K, E15Q, S87D, S87E, C125A, and C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) three amino acid substitutions of E15K, S87D or S87E, and C125A or C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) three amino acid substitutions of E15K, S87D or S87E, and C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) three amino acid substitutions of E15K, S87D, and C125S.

In still another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) three amino acid substitutions of E15K, S87E, and C125S.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) four amino acid substitutions at positions 3, 15, 87, and 125 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) four amino acid substitutions at positions T3, E15, S87, and C125 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) four amino acid substitutions of T3A, E15K or E15Q, S87D or S87E, and C125A or C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) four amino acid substitutions: T3A, E15K, S87D, and C125S.

In still another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) four amino acid substitutions: T3A, E15K, S87E, and C125S.

In one embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position 18 and an amino acid substitution at position 3, 15, 87, or 126 as set forth in SEQ ID NO: 1.

In another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution at position L18 and an amino acid substitution at position T3, E15, S87, or Q126 as set forth in SEQ ID NO: 1.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of L18C and an amino acid substitution of T3A, E15K, E15Q, S87D, S87E, or Q126E.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of L18C and an amino acid substitution of T3A, E15K, S87D, S87E, or Q126E.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) amino acid substitutions of L18C and Q126E.

In still another embodiment, the IL-2 domain in the fusion protein provided herein comprises (i) a replacement of the amino acid residues from positions 29 to 50 as set forth in SEQ ID NO: 1 with an IL-15 hinge fragment-containing peptide; and (ii) amino acid substitutions of T3A, L18C, and Q126E.

In certain embodiments, the IL-15 hinge fragment-containing peptide in the fusion protein provided herein comprises an amino acid sequence of one of SEQ ID NOs: 63 to 72. In certain embodiments, the IL-15 hinge fragment-containing peptide the fusion protein provided herein comprises an amino acid sequence of SEQ ID NO: 63. In certain embodiments, the IL-15 hinge fragment-containing peptide the fusion protein provided herein comprises an amino acid sequence of SEQ ID NO: 64. In certain embodiments, the IL-15 hinge fragment-containing peptide the fusion protein provided herein comprises an amino acid sequence of SEQ ID NO: 65. In certain embodiments, the IL-15 hinge fragment-containing peptide the fusion protein provided herein comprises an amino acid sequence of SEQ ID NO: 66. In certain embodiments, the IL-15 hinge fragment-containing peptide the fusion protein provided herein comprises an amino acid sequence of SEQ ID NO: 67. In certain embodiments, the IL-15 hinge fragment-containing peptide the fusion protein provided herein comprises an amino acid sequence of SEQ ID NO: 68. In certain embodiments, the IL-15 hinge fragment-containing peptide the fusion protein provided herein comprises an amino acid sequence of SEQ ID NO: 69. In certain embodiments, the IL-15 hinge fragment-containing peptide the fusion protein provided herein comprises an amino acid sequence of SEQ ID NO: 70. In certain embodiments, the IL-15 hinge fragment-containing peptide the fusion protein provided herein comprises an amino acid sequence of SEQ ID NO: 71. In certain embodiments, the IL-15 hinge fragment-containing peptide the fusion protein provided herein comprises an amino acid sequence of SEQ ID NO: 72.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) an amino acid substitution of T3A, E15K, L18C, S87D, S87E, C125S, or Q126E.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) an amino acid substitution of T3A, E15K, L18C, S87D, S87E, C125S, or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) an amino acid substitution of T3A, E15K, L18C, S87D, S87E, C125S, or Q126E.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) an amino acid substitution of T3A, E15K, L18C, S87D, S87E, C125S, or Q126E.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) an amino acid substitution of T3A, E15K, S87D, S87E, or C125S.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) an amino acid substitution of T3A, E15K, S87D, S87E, or C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) an amino acid substitution of T3A, E15K, S87D, S87E, or C125S.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) an amino acid substitution of T3A, E15K, S87D, S87E, or C125S.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) two amino acid substitutions, each independently selected from T3A, E15K, S87D, S87E, and C125S.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) two amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) two amino acid substitutions, each independently selected from T3A, E15K, S87D, S87E, and C125S.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) two amino acid substitutions, each independently selected from T3A, E15K, S87D, S87E, and C125S.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) three amino acid substitutions, each independently selected from T3A, E15K, S87D, S87E, and C125S.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) three amino acid substitutions, each independently selected from T3A, E15K, S87D, S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) three amino acid substitutions, each independently selected from T3A, E15K, S87D, S87E, and C125S.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) three amino acid substitutions, each independently selected from T3A, E15K, S87D, S87E, and C125S.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) four amino acid substitutions of T3A, E15K, S87D or S87E, and C125S.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) four amino acid substitutions of T3A, E15K, S87D or S87E, and C125S.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) four amino acid substitutions of T3A, E15K, S87D or S87E, and C125S.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) four amino acid substitutions of T3A, E15K, S87D or S87E, and C125S.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) an amino acid substitution of L18C and an amino acid substitution of T3A or Q126E.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) an amino acid substitution of L18C and an amino acid substitution of T3A or Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) an amino acid substitution of L18C and an amino acid substitution of T3A or Q126E.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) an amino acid substitution of L18C and an amino acid substitution of T3A or Q126E.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) amino acid substitutions of L18C and Q126E.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) amino acid substitutions of L18C and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) amino acid substitutions of L18C and Q126E.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) amino acid substitutions of L18C and Q126E.

In one embodiment, the IL-2 domain in the fusion protein provided herein comprises (i) a replacement of the amino acid residues from positions 29 to 50 of SEQ ID NO: 62 with an IL-15 hinge fragment-containing peptide of any one of SEQ ID NOs: 63 to 72; and (ii) amino acid substitutions of T3A, L18C, and Q126E.

In another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 63; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 64; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 65; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 66; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 67; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 68; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 69; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 70; and (ii) amino acid substitutions of T3A, L18C, and Q126E.

In yet another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 71; and (ii) amino acid substitutions of T3A, L18C, and Q126E.

In still another embodiment, the IL-2 domain in the fusion protein provided herein comprises (i) a replacement of the amino acid residues from positions 29 to 50 of SEQ ID NO: 62 with an IL-15 hinge fragment-containing peptide of SEQ ID NO: 72; and (ii) amino acid substitutions of T3A, L18C, and Q126E.

In one embodiment, the IL-2 domain in the fusion protein provided herein comprises the amino acid sequence of any one of SEQ ID NOs: 73 to 88, 118, and 119. In another embodiment, the IL-2 domain in the fusion protein provided herein comprises the amino acid sequence of SEQ ID NO: 73. In yet another embodiment, the IL-2 domain in the fusion protein provided herein comprises the amino acid sequence of SEQ ID NO: 74. In yet another embodiment, the IL-2 domain in the fusion protein provided herein comprises the amino acid sequence of SEQ ID NO: 75. In yet another embodiment, the IL-2 domain in the fusion protein provided herein comprises the amino acid sequence of SEQ ID NO: 76. In yet another embodiment, the IL-2 domain in the fusion protein provided herein comprises the amino acid sequence of SEQ ID NO: 77. In yet another embodiment, the IL-2 domain in the fusion protein provided herein comprises the amino acid sequence of SEQ ID NO: 78. In yet another embodiment, the IL-2 domain in the fusion protein provided herein comprises the amino acid sequence of SEQ ID NO: 79. In yet another embodiment, the IL-2 domain in the fusion protein provided herein comprises the amino acid sequence of SEQ ID NO: 80. In yet another embodiment, the IL-2 domain in the fusion protein provided herein comprises the amino acid sequence of SEQ ID NO: 81. In yet another embodiment, the IL-2 domain in the fusion protein provided herein comprises the amino acid sequence of SEQ ID NO: 82. In yet another embodiment, the IL-2 domain in the fusion protein provided herein comprises the amino acid sequence of SEQ ID NO: 83. In yet another embodiment, the IL-2 domain in the fusion protein provided herein comprises the amino acid sequence of SEQ ID NO: 84. In yet another embodiment, the IL-2 domain in the fusion protein provided herein comprises the amino acid sequence of SEQ ID NO: 85. In yet another embodiment, the IL-2 domain in the fusion protein provided herein comprises the amino acid sequence of SEQ ID NO: 86. In yet another embodiment, the IL-2 domain in the fusion protein provided herein comprises the amino acid sequence of SEQ ID NO: 87. In yet another embodiment, the IL-2 domain in the fusion protein provided herein comprises the amino acid sequence of SEQ ID NO: 88. In yet another embodiment, the IL-2 domain in the fusion protein provided herein comprises the amino acid sequence of SEQ ID NO: 118. In still another embodiment, the IL-2 domain in the fusion protein provided herein comprises the amino acid sequence of SEQ ID NO: 119.

In one embodiment, the sdAb comprises (i) CDR1 of SEQ ID NO: 6, CDR2 of SEQ ID NO: 7, and CDR3 of SEQ ID NO: 8; or (ii) CDR1 of SEQ ID NO: 14, CDR2 of SEQ ID NO: 15, and CDR3 of SEQ ID NO: 16. In one embodiment, the sdAb comprises CDR1 of SEQ ID NO: 6, CDR2 of SEQ ID NO: 7, and CDR3 of SEQ ID NO: 8. In another embodiment, the sdAb comprises CDR1 of SEQ ID NO: 14, CDR2 of SEQ ID NO: 15, and CDR3 of SEQ ID NO: 16.

In one embodiment, the sdAb has the structure of FR1-CDR1-FR2-CDR2-FR3-CDR3-FR4, wherein:
CDR1, CDR2, and CDR3 are:
   (i) CDR1 of SEQ ID NO: 6, CDR2 of SEQ ID NO: 7, and CDR3 of SEQ ID NO: 8; or
   (ii) CDR1 of SEQ ID NO: 14, CDR2 of SEQ ID NO: 15, and CDR3 of SEQ ID NO: 16;
FR1 is an amino acid sequence of SEQ ID NO: 9 or 17;
FR2 is an amino acid sequence of SEQ ID NO: 10 or 18;
FR3 is an amino acid sequence of SEQ ID NO: 11; and
FR4 is an amino acid sequence of SEQ ID NO: 12 or 19.

In another embodiment, the sdAb has the structure of FR1-CDR1-FR2-CDR2-FR3-CDR3-FR4, wherein:
CDR1, CDR2, and CDR3 are:
   (i) CDR1 of SEQ ID NO: 6, CDR2 of SEQ ID NO: 7, and CDR3 of SEQ ID NO: 8; or
   (ii) CDR1 of SEQ ID NO: 14, CDR2 of SEQ ID NO: 15, and CDR3 of SEQ ID NO: 16;
FR1 is an amino acid sequence of SEQ ID NO: 9;
FR2 is an amino acid sequence of SEQ ID NO: 10;
FR3 is an amino acid sequence of SEQ ID NO: 11; and
FR4 is an amino acid sequence of SEQ ID NO: 12.

In yet another embodiment, the sdAb has the structure of FR1-CDR1-FR2-CDR2-FR3-CDR3-FR4, wherein:
CDR1, CDR2, and CDR3 are:
   (i) CDR1 of SEQ ID NO: 6, CDR2 of SEQ ID NO: 7, and CDR3 of SEQ ID NO: 8; or
   (ii) CDR1 of SEQ ID NO: 14, CDR2 of SEQ ID NO: 15, and CDR3 of SEQ ID NO: 16;
FR1 is an amino acid sequence of SEQ ID NO: 17;
FR2 is an amino acid sequence of SEQ ID NO: 18;
FR3 is an amino acid sequence of SEQ ID NO: 11; and
FR4 is an amino acid sequence of SEQ ID NO: 19.

In one embodiment, the sdAb has an amino acid sequence of SEQ ID NO: 13 or 20. In another embodiment, the sdAb has an amino acid sequence of SEQ ID NO: 13. In yet another embodiment, the sdAb has an amino acid sequence of SEQ ID NO: 20.

In certain embodiments, the sdAb is a human antibody. In certain embodiments, the sdAb is a humanized antibody.

In certain embodiments, the sdAb binds to an HSA with a K_{d} ranging from about 10 pM to about 1,000 nM. In certain embodiments, the sdAb binds to an HSA with a K_{d} ranging from about 1 nM to about 500 nM. In certain embodiments, the sdAb binds to an HSA with a K_{d} ranging from about 1 nM to about 200 nM. In certain embodiments, the sdAb binds to an HSA with a K_{d} ranging from about 1 nM to about 100 nM.

In yet another embodiment, the sdAb is a V_{H}H sdAb that binds to an HSA.

In one embodiment, the V_{H}H sdAb comprises (i) heavy chain CDR1 of SEQ ID NO: 6, heavy chain CDR2 of SEQ ID NO: 7, and heavy chain CDR3 of SEQ ID NO: 8; or (ii) heavy chain CDR1 of SEQ ID NO: 14, heavy chain CDR2 of SEQ ID NO: 15, and heavy chain CDR3 of SEQ ID NO: 16. In one embodiment, the V_{H}H sdAb comprises heavy chain CDR1 of SEQ ID NO: 6, heavy chain CDR2 of SEQ ID NO: 7, and heavy chain CDR3 of SEQ ID NO: 8. In another embodiment, the V_{H}H sdAb comprises heavy chain CDR1 of SEQ ID NO: 14, heavy chain CDR2 of SEQ ID NO: 15, and heavy chain CDR3 of SEQ ID NO: 16.

In one embodiment, the V_{H}H sdAb has the structure of FR1-CDR1-FR2-CDR2-FR3-CDR3-FR4, wherein:
CDR1, CDR2, and CDR3 are:
   (i) CDR1 of SEQ ID NO: 6, CDR2 of SEQ ID NO: 7, and CDR3 of SEQ ID NO: 8; or
   (ii) CDR1 of SEQ ID NO: 14, CDR2 of SEQ ID NO: 15, and CDR3 of SEQ ID NO: 16;
FR1 is an amino acid sequence of SEQ ID NO: 9 or 17;
FR2 is an amino acid sequence of SEQ ID NO: 10 or 18;
FR3 is an amino acid sequence of SEQ ID NO: 11; and
FR4 is an amino acid sequence of SEQ ID NO: 12 or 19.

In another embodiment, the V_{H}H sdAb has the structure of FR1-CDR1-FR2-CDR2-FR3-CDR3-FR4, wherein:
CDR1, CDR2, and CDR3 are:
   (i) CDR1 of SEQ ID NO: 6, CDR2 of SEQ ID NO: 7, and CDR3 of SEQ ID NO: 8; or
   (ii) CDR1 of SEQ ID NO: 14, CDR2 of SEQ ID NO: 15, and CDR3 of SEQ ID NO: 16;
FR1 is an amino acid sequence of SEQ ID NO: 9;
FR2 is an amino acid sequence of SEQ ID NO: 10;
FR3 is an amino acid sequence of SEQ ID NO: 11; and
FR4 is an amino acid sequence of SEQ ID NO: 12.

In yet another embodiment, the V_{H}H sdAb has the structure of FR1-CDR1-FR2-CDR2-FR3-CDR3-FR4, wherein:
CDR1, CDR2, and CDR3 are:
   (i) CDR1 of SEQ ID NO: 6, CDR2 of SEQ ID NO: 7, and CDR3 of SEQ ID NO: 8; or
   (ii) CDR1 of SEQ ID NO: 14, CDR2 of SEQ ID NO: 15, and CDR3 of SEQ ID NO: 16;
FR1 is an amino acid sequence of SEQ ID NO: 17;
FR2 is an amino acid sequence of SEQ ID NO: 18;
FR3 is an amino acid sequence of SEQ ID NO: 11; and
FR4 is an amino acid sequence of SEQ ID NO: 19.

In one embodiment, the V_{H}H sdAb has an amino acid sequence of SEQ ID NO: 13 or 20. In another embodiment, the V_{H}H sdAb has an amino acid sequence of SEQ ID NO: 13. In yet another embodiment, the V_{H}H sdAb has an amino acid sequence of SEQ ID NO: 20.

In certain embodiments, the V_{H}H sdAb is a human antibody. In certain embodiments, the V_{H}H sdAb is a humanized antibody.

In certain embodiments, the V_{H}H sdAb binds to an HSA with a K_{d} ranging from about 10 pM to about 1,000 nM. In certain embodiments, the V_{H}H sdAb binds to an HSA with a K_{d} ranging from about 1 nM to about 500 nM. In certain embodiments, the V_{H}H sdAb binds to an HSA with a K_{d} ranging from about 1 nM to about 200 nM. In certain embodiments, the V_{H}H sdAb binds to an HSA with a K_{d} ranging from about 1 nM to about 100 nM.

In one embodiment, the peptide linker has an amino acid sequence of GSG or one of SEQ ID NOs: 21 to 61. In another embodiment, the peptide linker has an amino acid sequence of GSG or SEQ ID NO: 21, 22, or 23. In yet another embodiment, the peptide linker has an amino acid sequence of SEQ ID NO: 24, 25, 26, or 27. In yet another embodiment, the peptide linker has an amino acid sequence of SEQ ID NO: 28, 29, 30, or 31. In yet another embodiment, the peptide linker has an amino acid sequence of SEQ ID NO: 30. In yet another embodiment, the peptide linker has an amino acid sequence of SEQ ID NO: 32, 33, 34, or 35. In yet another embodiment, the peptide linker has an amino acid sequence of SEQ ID NO: 36, 37, 38, or 39. In yet another embodiment, the peptide linker has an amino acid sequence of SEQ ID NO: 40, 41, 42, or 43. In yet another embodiment, the peptide linker has an amino acid sequence of SEQ ID NO: 44, 45, 46, or 47. In yet another embodiment, the peptide linker has an amino acid sequence of SEQ ID NO: 48, 49, 50, or 51. In yet another embodiment, the peptide linker has an amino acid sequence of SEQ ID NO: 52, 53, or 54. In yet another embodiment, the peptide linker has an amino acid sequence of SEQ ID NO: 55, 56, or 57. In yet another embodiment, the peptide linker has an amino acid sequence of SEQ ID NO: 58, 59, 60, or 61. In yet another embodiment, the peptide linker is a linker having an amino acid sequence of SEQ ID NO: 60. In yet another embodiment, the peptide linker is a linker having an amino acid sequence of SEQ ID NO: 61. In still another embodiment, the peptide linker is a linker having an amino acid sequence of SEQ ID NO: 30, 59, 60, or 61.

In one embodiment, the fusion protein provided herein comprises an IL-2 domain having an amino acid sequence of any one of SEQ ID NOs: 73 to 104, 118, and 119; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In another embodiment, the fusion protein provided herein comprises an IL-2 domain having an amino acid sequence of any one of SEQ ID NOs: 73 to 88; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In one embodiment, the fusion protein provided herein comprises an IL-2 domain having an amino acid sequence of SEQ ID NO: 73; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In another embodiment, the fusion protein provided herein comprises an IL-2 domain having an amino acid sequence of SEQ ID NO: 74; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 75; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 76; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 77; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 78; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 79; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 80; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 81; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 82; an anti-HSA V_{H}H sdAb having an amino acid sequence of SEQ ID NO: 13 or 20; and a peptide linker having an amino acid sequence of GSG or one of SEQ ID NOs: 21 to 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 83; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 84; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 85; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 86; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 87; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In still another embodiment, the fusion protein provided herein comprises an IL-2 domain having an amino acid sequence of SEQ ID NO: 88; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of any one of SEQ ID NOs: 89 to 104; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In one embodiment, the fusion protein provided herein comprises an IL-2 domain having an amino acid sequence of SEQ ID NO: 89; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In another embodiment, the fusion protein provided herein comprises an IL-2 domain having an amino acid sequence of SEQ ID NO: 90; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 91; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 92; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 93; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 94; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 95; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 96; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 97; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 98; an anti-HSA V_{H}H sdAb having an amino acid sequence of SEQ ID NO: 13 or 20; and a peptide linker having an amino acid sequence of GSG or one of SEQ ID NOs: 21 to 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 99; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 100; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 101; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 102; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In yet another embodiment, the fusion protein provided herein comprises an IL-2 domain having an amino acid sequence of SEQ ID NO: 103; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In still another embodiment, the fusion protein provided herein comprises an IL-2 domain having an amino acid sequence of SEQ ID NO: 104; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In still another embodiment, the fusion protein provided herein comprises an IL-2 domain having an amino acid sequence of SEQ ID NO: 118 or 119; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In one embodiment, the fusion protein provided herein comprises an IL-2 domain having an amino acid sequence of SEQ ID NO: 118; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In another embodiment, the fusion protein provided herein comprises an IL-2 domain having an amino acid sequence of SEQ ID NO: 119; an anti-HSA V_{H}H sdAb having an amino acid sequence of SEQ ID NO: 13 or 20; and a peptide linker having an amino acid sequence of GSG or one of SEQ ID NOs: 21 to 61, in one embodiment, SEQ ID NO: 30, 59, 60, or 61; wherein the C-terminus of the anti-HSA V_{H}H sdAb is connected to the N-terminus of the IL-2 domain via the peptide linker.

In one embodiment, the fusion protein provided herein comprises an amino acid sequence of any one of SEQ ID NOs: 106 to 117 and 120 to 125. In another embodiment, the fusion protein provided herein comprises an amino acid sequence of SEQ ID NO: 106. In yet another embodiment, the fusion protein provided herein comprises an amino acid sequence of SEQ ID NO: 107. In yet another embodiment, the fusion protein provided herein comprises an amino acid sequence of SEQ ID NO: 108. In yet another embodiment, the fusion protein provided herein comprises an amino acid sequence of SEQ ID NO: 109. In yet another embodiment, the fusion protein provided herein comprises an amino acid sequence of SEQ ID NO: 110. In yet another embodiment, the fusion protein provided herein comprises an amino acid sequence of SEQ ID NO: 111. In yet another embodiment, the fusion protein provided herein comprises an amino acid sequence of SEQ ID NO: 112. In yet another embodiment, the fusion protein provided herein comprises an amino acid sequence of SEQ ID NO: 113. In yet another embodiment, the fusion protein provided herein comprises an amino acid sequence of SEQ ID NO: 114. In yet another embodiment, the fusion protein provided herein comprises an amino acid sequence of SEQ ID NO: 115. In yet another embodiment, the fusion protein provided herein comprises an amino acid sequence of SEQ ID NO: 116. In yet another embodiment, the fusion protein provided herein comprises an amino acid sequence of SEQ ID NO: 117. In yet another embodiment, the fusion protein provided herein comprises an amino acid sequence of SEQ ID NO: 120. In yet another embodiment, the fusion protein provided herein comprises an amino acid sequence of SEQ ID NO: 121. In yet another embodiment, the fusion protein provided herein comprises an amino acid sequence of SEQ ID NO: 122. In yet another embodiment, the fusion protein provided herein comprises an amino acid sequence of SEQ ID NO: 123. In yet another embodiment, the fusion protein provided herein comprises an amino acid sequence of SEQ ID NO: 124. In still another embodiment, the fusion protein provided herein comprises an amino acid sequence of SEQ ID NO: 125.

In certain embodiments, the fusion protein provided herein has a reduced binding affinity to an IL-2Rα as compared to the wild-type interleukin-2 described herein, e.g., of SEQ ID NO: 1. In certain embodiment, the binding affinity of the fusion protein provided herein to an IL-2Rα is measured by its K_{d}, which is the inverse of its K_{d}.

In certain embodiments, the fusion protein provided herein has a K_{d} to the IL-2Rα of no less than about 2 times, no less than about 5 times, no less than about 10 times, no less than about 100 times, or no less than about 1,000 times higher than that of the wild-type interleukin-2 to the IL-2Rα. In one embodiment, the fusion protein provided herein has a K_{d} to the IL-2Rα of no less than about 2 times higher than that of the wild-type interleukin-2 to the IL-2Rα. In another embodiment, the fusion protein provided herein has a K_{d} to the IL-2Rα of no less than about 5 times higher than that of the wild-type interleukin-2 to the IL-2Rα. In yet another embodiment, the fusion protein provided herein has a K_{d} to the IL-2Rα of no less than about 10 times higher than that of the wild-type interleukin-2 to the IL-2Rα. In yet another embodiment, the fusion protein provided herein has a K_{d} to the IL-2Rα of no less than about 100 times higher than that of the wild-type interleukin-2 to the IL-2Rα. In still another embodiment, the fusion protein provided herein has a K_{d} to the IL-2Rα of no less than about 1,000 times higher than that of the wild-type interleukin-2 to the IL-2Rα.

In certain embodiments, the fusion protein provided herein has a K_{d} to an IL-2Rα of no less than about 20 nM, no less than about 50 nM, no less than about 100 nM, no less than about 1 pM, no less than about 10 pM, no less than about 100 pM, or no less than about 1 mM. In certain embodiments, the fusion protein provided herein has a K_{d} to the IL-2Rα of no less than about 20 nM. In certain embodiments, the fusion protein provided herein has a K_{d} to the IL-2Rα of no less than about 50 nM. In certain embodiments, the fusion protein provided herein has a K_{d} to the IL-2Rα of no less than about 100 nM. In certain embodiments, the fusion protein provided herein has a K_{d} to the IL-2Rα of no less than about 1 pM. In certain embodiments, the fusion protein provided herein has a K_{d} to the IL-2Rα of no less than about 10 pM. In certain embodiments, the fusion protein provided herein has a K_{d} to the IL-2Rα of no less than about 100 pM. In certain embodiments, the fusion protein provided herein has a K_{d} to the IL-2Rα of no less than about 1 mM. In certain embodiments, the fusion protein provided herein has no measurable binding to the IL-2Rα. In certain embodiments, the fusion protein provided herein has no detectable binding to the IL-2Rα as measured with a surface plasmon resonance (SPR) method. In certain embodiments, the fusion protein provided herein has no detectable binding to the IL-2Rα as measured with bio-layer interferometry (BLI).

In yet another embodiment, the fusion protein provided herein has a selectivity for an IL-2Rβ over an IL-2Rα; wherein the selectivity is no greater than about 1, no greater than about 0.5, no greater than about 0.2, no greater than about 0.1, no greater than about 0.01, or no greater than about 0.001; and wherein the selectivity is measured as a ratio of a K_{d} of the fusion protein to the IL-2Rβ over a K_{d} of the fusion protein to the IL-2Rα. In certain embodiments, the fusion protein provided herein has a selectivity of no greater than about 1. In certain embodiments, the fusion protein provided herein has a selectivity of no greater than about 0.5. In certain embodiments, the fusion protein provided herein has a selectivity of no greater than about 0.2. In certain embodiments, the fusion protein provided herein has a selectivity of no greater than about 0.1. In certain embodiments, the fusion protein provided herein has a selectivity of no greater than about 0.01. In certain embodiments, the fusion protein provided herein has a selectivity of no greater than about 0.001.

In yet another embodiment, the fusion protein provided herein has a selectivity for an IL-2Rβ/γ complex over an IL-2Rα; wherein the selectivity is no greater than about 0.01 or no greater than about 0.001; and wherein the selectivity is measured as a ratio of a K_{d} of the fusion protein to the IL-2Rβ/γ complex over a K_{d} of the fusion protein to the IL-2Rα. In certain embodiments, the fusion protein provided herein has a selectivity of no greater than about 0.01. In certain embodiments, the fusion protein provided herein has a selectivity of no greater than about 0.001. In one embodiment, the dissociation constants of an IL-2 to the IL-2Rα and the IL-2Rβ/γ complex are determined as described in Richert et al., J. Mol. Biol. 2004, 339, 1115-1119.

In one embodiment, the IL-2Rα is a human IL-2Rα. In another embodiment, the human IL-2Rα has an amino acid sequence of SEQ ID NO: 3.

In one embodiment, the IL-2Rβ is a human IL-2Rβ. In another embodiment, the human IL-2Rβ has an amino acid sequence of SEQ ID NO: 4.

In one embodiment, the IL-2Rγ is a human IL-2Rγ. In another embodiment, the human IL-2Rγ has an amino acid sequence of SEQ ID NO: 5.

In one embodiment, a K_{d} of an IL-2 to an IL-2Rα is determined with a surface plasmon resonance (SPR) method. In another embodiment, a K_{d} of an IL-2 to an IL-2Rα is determined with a BIACORE® assay. In yet another embodiment, a K_{d} of an IL-2 to an IL-2Rα is determined with bio-layer interferometry (BLI). In still another embodiment, a K_{d} of an IL-2 to an IL-2Rα is determined with an OCTET® assay.

In one embodiment, a K_{d} of an IL-2 to an IL-2Rβ is determined with an SPR method. In another embodiment, a K_{d} of an IL-2 to an IL-2Rβ is determined with a BIACORE® assay. In yet another embodiment, a K_{d} of an IL-2 to an IL-2Rβ is determined with BLI. In still another embodiment, a K_{d} of an IL-2 to an IL-2Rβ is determined with an OCTET® assay.

In one embodiment, a K_{d} of an IL-2 to an IL-2Rβ/γ complex is determined with an SPR method. In another embodiment, a K_{d} of an IL-2 to an IL-2Rβ/γ complex is determined with a BIACORE® assay. In yet another embodiment, a K_{d} of an IL-2 to an IL-2Rβ/γ complex is determined with BLI. In still another embodiment, a K_{d} of an IL-2 to an IL-2Rβ/γ complex is determined with an OCTET® assay.

In one embodiment, a K_{d} of an IL-2 to an IL-2Rα/β/γ complex is determined with an SPR method. In another embodiment, a K_{d} of an IL-2 to an IL-2Rα/β/γ complex is determined with a BIACORE® assay. In yet another embodiment, a K_{d} of an IL-2 to an IL-2Rα/β/γ complex is determined with BLI. In still another embodiment, a K_{d} of an IL-2 to an IL-2Rα/β/γ complex is determined with an OCTET® assay.

In one embodiment, the fusion protein provided herein is an isolated fusion protein. In another embodiment, the fusion protein provided herein is a recombinant fusion protein.

In one embodiment, the fusion protein provided herein is N-glycosylated. In another embodiment, the fusion protein provided herein is N-glycosylated at a glycosylation site.

In one embodiment, the fusion protein provided herein has one glycan. In another embodiment, the fusion protein provided herein has one glycan attached to the nitrogen in the side chain of an asparagine residue.

In one embodiment, the fusion protein provided herein has two glycans. In another embodiment, the fusion protein provided herein has two glycans, of which at least one glycan is attached to the nitrogen in the side chain of an asparagine residue. In yet another embodiment, the fusion protein provided herein has two glycans, each of which is attached to the nitrogen in the side chain of an asparagine residue.

In one embodiment, the fusion protein provided herein has three glycans. In one embodiment, the glycan is an N-glycan.

In one embodiment, the N-glycan on the fusion protein provided herein is oligomannose-type. In another embodiment, the N-glycan on the fusion protein provided herein is complex-type. In another embodiment, the N-glycan on the fusion protein provided herein is hybrid-type.

In one embodiment, the N-glycan on the fusion protein provided herein is biantennary complex-type. In another embodiment, the N-glycan on the fusion protein provided herein is triantennary complex-type. In yet another embodiment, the N-glycan on the fusion protein provided herein is tetraantennary complex-type.

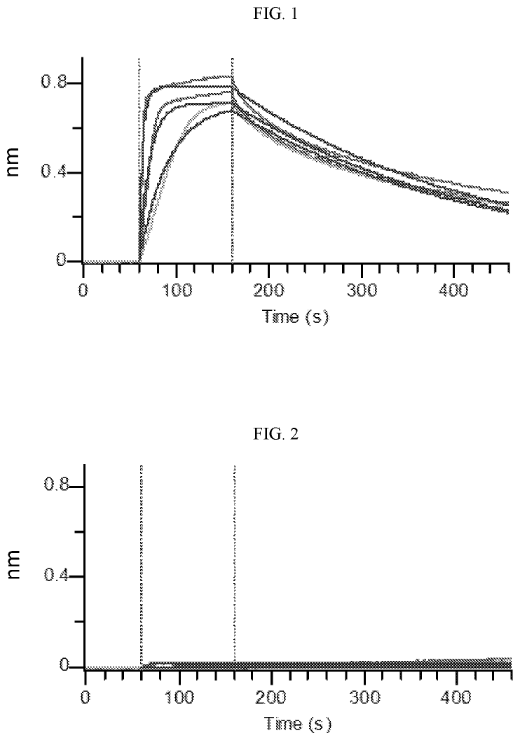

In one embodiment, the N-glycan on the fusion protein provided herein is one of the glycans described in FIG. 1. Szabo et al., J. Proteome. Res. 2018, 17, 1559-1574, the disclosure of which is incorporated herein by reference in its entirety.

In one embodiment, the fusion protein provided herein is produced from a yeast cell, insect cell, mammalian cell, a human cell, or a plant cell. In another embodiment, the fusion protein provided herein is produced from a yeast cell. In yet another embodiment, the fusion protein provided herein is produced from an insect cell. In yet another embodiment, the fusion protein provided herein is produced from a mammalian cell. In yet another embodiment, the fusion protein provided herein is produced from a CHO cell. In yet another embodiment, the fusion protein provided herein is produced from a human cell. In still another embodiment, the fusion protein provided herein is produced from a plant cell.

In certain embodiments, the fusion protein provided herein further includes one or more additional substitutions, deletions, and/or insertions.

In certain embodiments, the fusion protein provided herein comprises an amino acid substitution, deletion, and/or insertion that results in an enhanced stability and/or production yield. In certain embodiments, the fusion protein provided herein comprises an amino acid substitution, deletion, and/or insertion that results in a reduced binding affinity to an IL-2Rα. In certain embodiments, the fusion protein provided herein comprises an amino acid substitution, deletion, and/or insertion that results in a reduced binding affinity to an IL-2Rβ. In certain embodiments, the fusion protein provided herein comprises an amino acid substitution, deletion, and/or insertion that results in an enhanced binding affinity to an IL-2Rβ. In certain embodiments, the fusion protein provided herein comprises an amino acid substitution, deletion, and/or insertion that results in a reduced binding affinity to an IL-2Rγ. In certain embodiments, the fusion protein provided herein comprises an amino acid substitution, deletion, and/or insertion that results in a reduced binding affinity to an IL-2Rβ/γ complex.

In certain embodiments, the fusion protein provided herein comprises an amino acid substitution, deletion, and/or insertion that results in (i) an enhanced stability and/or production yield and (ii) a reduced binding affinity to an IL-2Rα. In certain embodiments, the fusion protein provided herein comprises an amino acid substitution, deletion, and/or insertion that results in (i) an enhanced stability and/or production yield and (ii) a reduced binding affinity to an IL-2Rβ. In certain embodiments, the fusion protein provided herein comprises an amino acid substitution, deletion, and/or insertion that results in (i) an enhanced stability and/or production yield and (ii) an enhanced binding affinity to an IL-2Rβ. In certain embodiments, the fusion protein provided herein comprises an amino acid substitution, deletion, and/or insertion that results in (i) an enhanced stability and/or production yield and (ii) a reduced binding affinity to an IL-2Rγ. In certain embodiments, the fusion protein provided herein comprises an amino acid substitution, deletion, and/or insertion that results in (i) an enhanced stability and/or production yield and (ii) a reduced binding affinity to an IL-2Rβ/γ complex.

In certain embodiments, the fusion protein provided herein comprises an amino acid substitution, deletion, and/or insertion that results in (i) a reduced binding affinity to an IL-2Rα and (ii) a reduced binding affinity to an IL-2Rβ. In certain embodiments, the fusion protein provided herein comprises an amino acid substitution, deletion, and/or insertion that results in (i) a reduced binding affinity to an IL-2Rα and (ii) an enhanced binding affinity to an IL-2Rβ. In certain embodiments, the fusion protein provided herein comprises an amino acid substitution, deletion, and/or insertion that results in (i) a reduced binding affinity to an IL-2Rα and (ii) a reduced binding affinity to an IL-2Rγ. In certain embodiments, the fusion protein provided herein comprises an amino acid substitution, deletion, and/or insertion that results in (i) a reduced binding affinity to an IL-2Rα and (ii) a reduced binding affinity to an IL-2Rβ/γ complex.

In certain embodiments, the fusion protein provided herein comprises an amino acid substitution, deletion, and/or insertion that results in (i) an enhanced stability and/or production yield, (ii) a reduced binding affinity to an IL-2Rα, and (iii) a reduced binding affinity to an IL-2Rβ. In certain embodiments, the fusion protein provided herein comprises an amino acid substitution, deletion, and/or insertion that results in (i) an enhanced stability and/or production yield, (ii) a reduced binding affinity to an IL-2Rα, and (iii) an enhanced binding affinity to an IL-2Rβ. In certain embodiments, the fusion protein provided herein comprises an amino acid substitution, deletion, and/or insertion that results in (i) an enhanced stability and/or production yield, (ii) a reduced binding affinity to an IL-2Rα, and (iii) a reduced binding affinity to an IL-2Rγ In certain embodiments, the fusion protein provided herein comprises an amino acid substitution, deletion, and/or insertion that results in (i) an enhanced stability and/or production yield, (ii) a reduced binding affinity to an IL-2Rα, and (iii) a reduced binding affinity to an IL-2Rβ/γ complex.

### Pharmaceutical Compositions

In one embodiment, provided herein is a pharmaceutical composition comprising a fusion protein provided herein and a pharmaceutically acceptable excipient.

In one embodiment, the pharmaceutical composition is formulated as single dosage form.

In one embodiment, the pharmaceutical composition provided herein is a solid formulation. In another embodiment, the pharmaceutical composition provided herein is a lyophilized solid formulation. In yet another embodiment, the pharmaceutical composition provided herein is a solution. In still another embodiment, the pharmaceutical composition provided herein is an aqueous solution.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for intratumoral administration.

In one embodiment, the pharmaceutical composition provided herein comprises a fusion protein provided herein, mannitol, sodium dodecyl sulfate, monobasic sodium phosphate, dibasic sodium phosphate, and water.

In one embodiment, the pharmaceutical composition provided herein has a pH ranging from about 7.2 to about 7.8. In another embodiment, the pharmaceutical composition provided herein has a pH of about 7.5.

### Methods of Use

In one embodiment, provided herein is a method for treating, preventing, or ameliorating a proliferative disease in a subject, comprising administering to the subject in need thereof a therapeutically effective amount of a fusion protein provided herein.

In one embodiment, the proliferative disease is cancer. In another embodiment, the proliferative disease is melanoma, ovarian cancer, pancreatic cancer, or renal cell carcinoma (RCC). In yet another embodiment, the proliferative disease is melanoma. In yet another embodiment, the proliferative disease is ovarian cancer. In yet another embodiment, the proliferative disease is pancreatic cancer. In still another embodiment, the proliferative disease is RCC.

In one embodiment, the proliferative disease is metastatic cancer. In another embodiment, the proliferative disease is metastatic melanoma, metastatic ovarian cancer, metastatic pancreatic cancer, or metastatic RCC. In yet another embodiment, the proliferative disease is metastatic melanoma. In yet another embodiment, the proliferative disease is metastatic ovarian cancer. In yet another embodiment, the proliferative disease is metastatic pancreatic cancer. In still another embodiment, the proliferative disease is metastatic RCC.

In certain embodiments, the therapeutically effective amount of the fusion protein is ranging from about 0.001 to 100 mg per kg subject body weight per day (mg/kg per day), from about 0.01 to about 75 mg/kg per day, from about 0.1 to about 50 mg/kg per day, from about 0.5 to about 25 mg/kg per day, or from about 1 to about 20 mg/kg per day, which can be administered in single or multiple doses. Within this range, the dosage can be ranging from about 0.005 to about 0.05, from about 0.05 to about 0.5, from about 0.5 to about 5.0, from about 1 to about 15, from about 1 to about 20, or from about 1 to about 50 mg/kg per day.

In certain embodiments, the subject is a mammal. In certain embodiments, the subject is a human.

In one embodiment, provided herein is a method of activating an immune effector cell, comprising contacting the cell with an effective amount of a fusion protein provided herein.

The disclosure will be further understood by the following non-limiting examples.

### EXAMPLES

### Example 1

### Cloning, Expression, and Purification of Anti-HSA-hIL-2 Fusion Proteins

The protein sequence of the human IL-2 (hIL-2) was obtained from UniProt (P60568, 21-153 aa). An hTL-2 mutein was generated by introducing a mutation to attenuating CD25 binding. The deoxyoligonucleotide sequence of the hTL-2 mutein was codon optimized for CHO cell expression.

For a fusion protein comprising an anti-HSA V_{H}H sdAb and hIL-2 mutein, the C-terminus of the anti-HSA V_{H}H sdAb was fused to the N-terminus of the hIL-2 mutein with a peptide linker. The deoxyoligonucleotide sequences encoding the anti-HSA V_{H}H sdAb, hIL-2 mutein, and peptide linker were seamlessly assembled together by homology assembly cloning with a commercially available kit. The oligonucleotide of the fusion protein was inserted into UCOE® expression vector for CHO cell expression.

Each fusion protein produced in the CHO cells was purified by a two-step purification process: protein A affinity chromatography using protein A (e.g., AMSPHERE™ A3 or MABSELECT™ SURE™) resin, and ion exchange chromatography (e.g., CAPTO™ Q IMPRES or CAPTO™ S IMPACT) or hydrophobic interaction chromatography (e.g., Phenyl HP).

Anti-HSA-hIL-2 fusion proteins A1 to A6, A14, A15, and A17 were prepared. Anti-HSA-hIL-2 fusion proteins A7 to A12, A13, A16, and A18 are prepared.

### Example 2

### CD25 and CD122 Bindings of Anti-HSA-hIL-2 Fusion Proteins

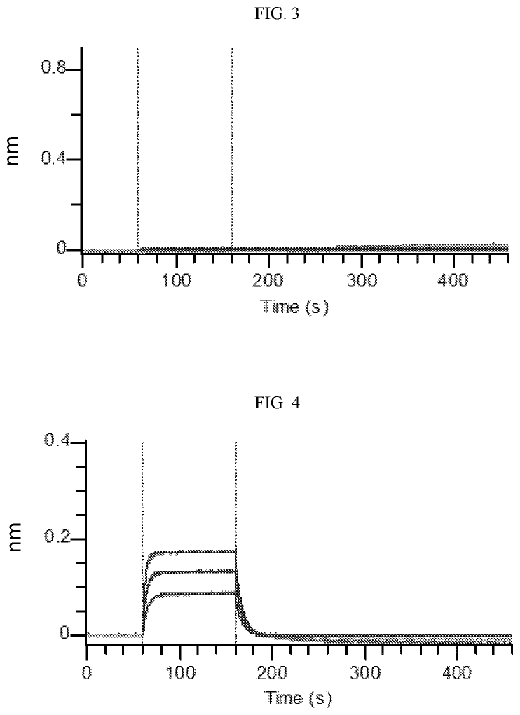

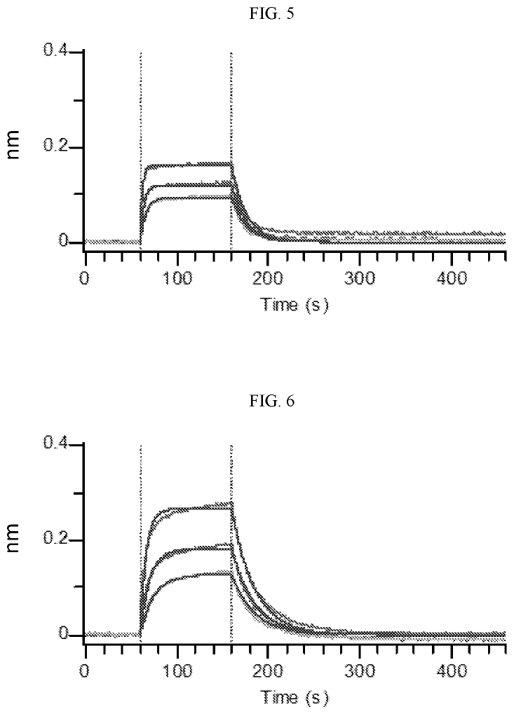

OCTET® RED96 was used to characterize the interactions of anti-HSA-hIL-2 fusion proteins with human IL-2Rα (CD25) and IL-2Rβ (CD122). Briefly, hIL-2Rα and hIL-2Rβ were each loaded onto a biosensor. Each biosensor was then dipped into a solution containing an anti-HSA-hIL-2 fusion protein at 10 to 2,000 nM. Primary experimental data was analyzed with global fitting to determine a binding affinity (K_{d}). The sensorgraphs are shown in FIGS. 1-6. The results are summarized in Table 1.

TABLE 1

| | | K_{d} (nM) | |
|---|---|---|---|
| Fusion Protein | IL-2 Mutein | hIL-2Rα | hIL-2Rβ |
| C1 | hIL-2 mutein (C125S) | 6.5 | 533 |
| (SEQ ID NO: 105) | (SEQ ID NO: 2) | | |
| A1 | hIL-2-hIL15 chimera 7 | NB^{a} | 55 |
| (SEQ ID NO: 106) | (SEQ ID NO: 79) | | |
| A2 | hIL-2-hIL15 chimera 12 | NB^{a} | 139 |
| (SEQ ID NO: 107) | (SEQ ID NO: 84) | | |
| A3 | hIL-2-hIL15 chimera 7 | NB^{a} | 25 |
| (SEQ ID NO: 108) | (SEQ ID NO: 79) | | |
| A4 | hIL-2-hIL15 chimera 12 | NB^{a} | 278 |
| (SEQ ID NO: 109) | (SEQ ID NO: 84) | | |
| A15 | hIL-2-hIL15 chimera 12 | NB^{a} | 94 |
| (SEQ ID NO: 122) | (SEQ ID NO: 84) | | |
| A17 | hIL-2-hIL15 chimera 12 | NB^{a} | 169 |
| (SEQ ID NO: 124) | (SEQ ID NO: 84) | | |

^{a}NB: no binding detected

### Example 3

### Effect of Anti-HSA-hIL-2 Fusion Proteins on STAT5 Signal Activation in CD3 T-Cells

In vitro potency of an anti-HSA-hIL-2 fusion protein was measured by quantifying phosphorylation of STAT5 in preactivated CD3 T-cells, which express hIL-2Rα, β, and γ chains. CD3 T-cells were isolated from human buffy coat using ROSETTESEP™ T cells isolation kit. Activated CD3 T-cells were prepared by incubating the CD3 T-cells in RPMI-1640 medium containing 10% fetal bovine serum with a CD3/CD28 T-cell activation mix and expanded with an IL-2 for 10 days, which were then frozen in liquid nitrogen for future use. Before the day of assay, the activated CD3 T cells were thawed and grown overnight in a RPMI medium containing 10% FBS. The activated cells (150,000) were stimulated with an anti-HSA-hIL-2 fusion protein for 20 min at 37° C. in 5% CO₂ in Hanks Balanced Salt Solution containing 10 mM HEPES. Phospho-STAT5 was measured using a phospho-STAT5 (Tyr694) homogeneous time resolved fluorescence (HTRF) assay. The signal ratio of 665 nm/620 nm was multiplied by 1,000, plotted, and fit using a dose response curve to calculate an EC₅₀. The results are summarized in Table 2 below.

TABLE 2

| Fusion Protein | IL-2 Mutein | EC₅₀ (nM) |
|---|---|---|
| A1 | hIL-2-hIL15 chimera 7 | 2.3 |
| (SEQ ID NO: 106) | (SEQ ID NO: 79) | |
| A2 | hIL-2-hIL15 chimera 12 | 7 |
| (SEQ ID NO: 107) | (SEQ ID NO: 84) | |
| A3 | hIL-2-hIL 15 chimera 7 | 1.6 |
| (SEQ ID NO: 108) | (SEQ ID NO: 79) | |
| A4 | hIL-2-hIL15 chimera 12 | 3.6 |
| (SEQ ID NO: 109) | (SEQ ID NO: 84) | |
| A14 | hIL-2-hIL15 chimera 34 | 40 |
| (SEQ ID NO: 121) | (SEQ ID NO: 119) | |
| A15 | hIL-2-hIL15 chimera 12 | 6.7 |
| (SEQ ID NO: 122) | (SEQ ID NO: 84) | |
| A17 | hIL-2-hIL15 chimera 12 | 1.5 |
| (SEQ ID NO: 124) | (SEQ ID NO: 84) | |

### Example 4

### Effect of Anti-HSA-hIL-2 Fusion Proteins on STAT5 Signal Activation in CTLL2 Cells

In vitro potency of an anti-HSA-hIL-2 fusion protein was measured by quantifying phosphorylation of STAT5 in CTLL2 cells. The CTLL2 cells were grown in RPMI-1640 medium containing 10% fetal bovine and 10% T-STIM with concanavalin A (IL-2 culture supplement). Before the day of assay, the CTLL2 cells were starved overnight in regular RPMI medium containing 10% FBS only. The CTLL2 cells (150,000) were stimulated with the anti-HSA-hIL-2 fusion protein for 20 min at 37 C in 5% CO₂ in Hanks Balanced Salt Solution containing 10 mM HEPES. Phospho-STAT5 was measured using a phospho-STAT5 (Tyr694) homogeneous time resolved fluorescence (HTRF) assay. The signal ratio of 665 nm/620 nm was multiplied by 1,000, plotted, and fit using a dose response curve to calculate an EC₅₀. The results are summarized in Table 3 below.

TABLE 3

| Fusion Protein | IL-2 Mutein | EC₅₀ (nM) |
|---|---|---|
| A1 | hIL-2-hIL15 chimera 7 | 3.6 |
| (SEQ ID NO: 106) | (SEQ ID NO: 79) | |
| A2 | hIL-2-hIL 15 chimera 12 | 8.2 |
| (SEQ ID NO: 107) | (SEQ ID NO: 84) | |
| A3 | hIL-2-hIL15 chimera 7 | 2 |
| (SEQ ID NO: 108) | (SEQ ID NO: 79) | |
| A4 | hIL-2-hIL15 chimera 12 | 2.7 |
| (SEQ ID NO: 109) | (SEQ ID NO: 84) | |
| A15 | hIL-2-hIL 15 chimera 12 | 17 |
| (SEQ ID NO: 122) | (SEQ ID NO: 84) | |

### Example 5

### In Vitro Cytotoxicity of IL-2-Anti-HSA Fusion Proteins

The in vitro potency of an anti-HSA-IL-2 fusion protein was determined by quantifying improvement in N87 (stomach cancer), CAPAN-2 (pancreatic adenocarcinoma), SKOV3 (ovarian cancer) cell killing by CD3/CD28 stimulated CD3+ T-cell. Cancer cells were maintained in RPMI-1640 containing 10% fetal bovine serum and penicillin/streptomycin. On Day 0, 5,000 cells/well were plated in the culture medium in a 96-well flat bottom plate. On Day 1, 15,000 CD3+ T cells/well and 1:300 diluted anti-CD3/anti-CD28 antibody complex (IMMUNOCULT™, 10971) were added to the cancer cells together with the anti-HSA-IL-2 fusion protein. The plates were incubated for 48 h at 37° C. in 5% CO₂. The cells were then fixed with 4% paraformaldehyde and nuclei were stained with SYTOX™ Orange. The number of remaining cancer cells was calculated by counting the number of cancer cell nuclei remaining in each well using a CYTATION™ 1 Cell Imaging Multi-Mode Reader. Lower cell counts indicated better CD3+ T-cell mediated cell killing. The IC₅₀ values determined are summarized in Table 4 below.

TABLE 4

| | | IC₅₀ (nM) | | |
|---|---|---|---|---|
| Fusion Protein | IL-2 | N87 | Capan-2 | SKOV3 |
| C1 | hIL-2 mutein (C125S) | 0.0038 | 0.00048 | 0.0015 |
| (SEQ ID NO: 105) | (SEQ ID NO: 2) | | | |
| A2 | hIL-2-hIL 15 | 0.16 | 0.037 | 0.95 |
| (SEQ ID NO: 107) | chimera 12 | | | |
| | (SEQ ID NO: 84) | | | |
| A4 | hIL-2-hIL15 | 0.14 | 0.041 | 1.5 |
| (SEQ ID NO: 109) | chimera 12 | | | |
| | (SEQ ID NO: 84) | | | |

### Example 6

### Antitumor Activity of IL-2-Anti-HSA Fusion Proteins in Xenograft Mouse Model for Colon Cancer

MC38 cells are cultured and maintained in DMEM media supplemented with 10% fetal bovine serum, GLUTAMAX™, non-essential amino acids (NEAA), sodium pyruvate, and penicillin/streptomycin. The cells are trypsinized, washed with the media, and counted. The cells are diluted with PBS and 5×10⁵ cells in PBS (50 μL) are injected subcutaneously into anesthetized C57BL/6 mice using an 18-gauge needle. A stock solution of an IL-2-anti-HSA fusion protein is diluted in PBS on the day of dosing and the mice are dosed intraperitoneally with PBS (control) or the IL-2-anti-HSA fusion protein in PBS twice a week for two weeks. Tumor sizes (length (L) and width (W)) are measured twice per week using a digital caliper, and the tumor volume is calculated (L×W×W)/2.

### Example 7

### Antitumor Activity of IL-2-Anti-HSA Fusion Proteins in Xenograft Mouse Model for Colorectal Cancer

CT26 cells are cultured and maintained in RPMI media supplemented with 10% fetal bovine serum, GLUTAMAX™, and penicillin/streptomycin. The cells are trypsinized, washed with media, and counted. The cells are diluted with PBS and 1×10⁶ cells in PBS (100 μL) are injected subcutaneously into anesthetized BALB/c mice using an 18-gauge needle. A stock solution of an IL-2-anti-HSA fusion protein is diluted in PBS on the day of dosing and the mice are dosed intraperitoneally with PBS (control) or the IL-2-anti-HSA fusion protein in PBS twice a week for two weeks. Tumor sizes (length (L) and width (W)) are measured twice per week using a digital caliper, and the tumor volume is calculated (L×W×W)/2.

### Example 8

### Antitumor Activity of IL-2-Anti-HSA Fusion Proteins in Xenograft Mouse Model for Colorectal Cancer

HT-29 cells are cultured and maintained in McCoys 5a media supplemented with 10% fetal bovine serum and penicillin/streptomycin. The cells are trypsinized, washed with media, counted, and washed with PBS. The cell suspension (1×10⁶ cells in PBS (100 μL)) is injected subcutaneously into anesthetized NSG or NCG mice using a 27-gauge needle. After 6 days, human PBMCs (1×10⁷ cells in PBS (100 μL)) are injected into the tail vein of each mouse. A stock solution of an IL-2-anti-HSA fusion protein is diluted in PBS on the day of dosing and the mice are dosed intraperitoneally with PBS (control) or the IL-2-anti-HSA fusion protein in PBS twice a week for two weeks. Tumor sizes (length (L) and width (W)) are measured twice per week using a digital caliper, and the tumor volume is calculated (L×W×W)/2.

### Example 9

### Antitumor Activity of IL-2-Anti-HSA Fusion Proteins in Xenograft Mouse Model for Gastric Carcinoma

NCI-N87 cells were cultured and maintained in RPMI media supplemented with 10% fetal bovine serum and penicillin/streptomycin. The cells are trypsinized, washed with media, counted, and washed with PBS. The cell suspension (3×10⁶ cells in PBS (100 μL)) is injected subcutaneously into anesthetized NSG or NCG mice using a 23-gauge needle. After 6 days, human PBMCs (1×10⁷ cells in PBS (100 μL)) are injected into the tail vein of each mouse. A stock solution of an IL-2-anti-HSA fusion protein is diluted in PBS on the day of dosing and the mice are dosed intraperitoneally with PBS (control) or the IL-2-anti-HSA fusion protein in PBS twice a week for two weeks. Tumor sizes (length (L) and width (W)) are measured twice per week using a digital caliper, and the tumor volume is calculated (L×W×W)/2.

### Example 10

### Glycan Analysis

The glycan profile of a fusion protein is analyzed using an ADVANCEBIO® GLY-X™ N-glycan prep with INSTANTPC™ kit. The fusion peptide is denatured and N-glycans are released by an N-glycanase at 50° C. The released N-glycans are labeled by an INSTANTPC™ dye and then cleaned up with a Gly-X™. The labeled glycans are analyzed on an HPLC system equipped with an ACQUITY UPLC Glycan BEH Amide column (130 Å, 1.7 μm, 2.1 mm×150 mm) connected to a Shimadzu Nexera-i LC-2040C 3D MT coupled with a RF-20A fluorescence detector. The N-glycans are identified by comparing them with the INSTANTPC™ labeled glycan standard libraries from Agilent Technologies.

Sequences described herein are provided in the sequence table below.

SEQUENCE TABLE

| SEQ | | |
|---|---|---|
| ID | | |
| NO: | Description | Amino Acid Sequence |
| 1 | hIL-2 | APTSSSTKKTQLQLEHLLLD |
| | | LQMILNGINNYKNPKLTRML |
| | | TFKFYMPKKATELKHLQCLE |
| | | EELKPLEEVLNLAQSKNFHL |
| | | RPRDLISNINVIVLELKGSE |
| | | TTFMCEYADETATIVEFLNR |
| | | WITFCQSIISTLT |
| 2 | hIL-2 mutein | APTSSSTKKTQLQLEHLLLD |
| | (C125S) | LQMILNGINNYKNPKLTRML |
| | | TFKFYMPKKATELKHLQCLE |
| | | EELKPLEEVLNLAQSKNFHL |
| | | RPRDLISNINVIVLELKGSE |
| | | TTFMCEYADETATIVEFLNR |
| | | WITFSQSIISTLT |
| 3 | hIL-2Rα | ELCDDDPPEIPHATFKAMAY |
| | | KEGTMLNCECKRGFRRIKSG |
| | | SLYMLCTGNSSHSSWDNQCQ |
| | | CTSSATRNTTKQVTPQPEEQ |
| | | KERKTTEMQSPMQPVDQASL |
| | | PGHCREPPPWENEATERIYH |
| | | FVVGQMVYYQCVQGYRALHR |
| | | GPAESVCKMTHGKTRWTQPQ |
| | | LICTGEMETSQFPGEEKPQA |
| | | SPEGRPESETSCLVTTTDFQ |
| | | IQTEMAATMETSIFTTEYQ |
| 4 | hIL-2Rβ | AVNGTSQFTCFYNSRANISC |
| | | VWSQDGALQDTSCQVHAWPD |
| | | RRRWNQTCELLPVSQASWAC |
| | | NLILGAPDSQKLTTVDIVTL |
| | | RVLCREGVRWRVMAIQDFKP |
| | | FENLRLMAPISLQVVHVETH |
| | | RCNISWEISQASHYFERHLE |
| | | FEARTLSPGHTWEEAPLLTL |
| | | KQKQEWICLETLTPDTQYEF |
| | | QVRVKPLQGEFTTWSPWSQP |
| | | LAFRTKPAALGKD |
| 5 | hIL-2Rγ | LNTTILTPNGNEDTTADFFL |
| | | TTMPTDSLSVSTLPLPEVQC |
| | | FVFNVEYMNCTWNSSSEPQP |
| | | TNLTLHYWYKNSDNDKVQKC |
| | | SHYLFSEEITSGCQLQKKEI |
| | | HLYQTFVVQLQDPREPRRQA |
| | | TQMLKLQNLVIPWAPENLTL |
| | | HKLSESQLELNWNNRFLNHC |
| | | LEHLVQYRTDWDHSWTEQSV |
| | | DYRHKFSLPSVDGQKRYTFR |
| | | VRSRFNPLCGSAQHWSEWSH |
| | | PIHWGSNTSKENPFLFALEA |
| 6 | Anti-HSA-1 CDR1 | GSTWSINT |
| 7 | Anti-HSA-1 CDR2 | ISSGGST |
| 8 | Anti-HSA-1 CDR3 | YAQSTWYPPS |
| 9 | Anti-HSA-1 FR1 | EVOLVESGGGLVQPGGSLRL |
| | | SCAAS |
| 10 | Anti-HSA-1 FR2 | LAWYRQAPGKQRDLVAR |
| 11 | Anti-HSA-1 FR3 | YYADSVKGRFTISRDNSKNT |
| | | LYLQMNSLRAEDTAVYYC |
| 12 | Anti-HSA-1 FR4 | WGQGTLVTVSS |
| 13 | Anti-HSA-1 | EVOLVESGGGLVQPGGSLRL |
| | | SCAASGSTWSINTLAWYRQA |
| | | PGKQRDLVARISSGGSTYYA |
| | | DSVKGRFTISRDNSKNTLYL |
| | | QMNSLRAEDTAVYYCYAQST |
| | | WYPPSWGQGTLVTVSS |
| 14 | Anti-HSA-2 CDR1 | GFAFRGFG |
| 15 | Anti-HSA-2 CDR2 | INNGGSDT |
| 16 | Anti-HSA-2 CDR3 | AIGGPGASP |
| 17 | Anti-HSA-2 FR1 | QVQLVESGGGVVQPGGSLRL |
| | | SCAAS |
| 18 | Anti-HSA-2 FR2 | MSWVRQAPGKGLEWVSS |
| 19 | Anti-HSA-2 FR4 | SGQGTQVTVSS |
| 20 | Anti-HSA-2 | QVQLVESGGGVVQPGGSLRL |
| | | SCAASGFAFRGFGMSWVRQA |
| | | PGKGLEWVSSINNGGSDTYY |
| | | ADSVKGRFTISRDNSKNTLY |
| | | LQMNSLRAEDTAVYYCAIGG |
| | | PGASPSGQGTQVTVSS |
| 21 | (GSG)₂ Linker | GSGGSG |
| 22 | (GSG)₃ Linker | GSGGSGGSG |
| 23 | (GSG)₄ Linker | GSGGSGGSGGSG |
| 24 | G3S Linker | GGGS |
| 25 | (G3S)₂ Linker | GGGSGGGS |
| 26 | (G3S)₃ Linker | GGGSGGGSGGGS |
| 27 | (G3S)₄ Linker | GGGSGGGSGGGSGGGS |
| 28 | G4S Linker | GGGGS |
| 29 | (G4S)₂ Linker | GGGGSGGGGS |
| 30 | (G4S)₃ Linker | GGGGSGGGGSGGGGS |
| 31 | (G4S)₄ Linker | GGGGSGGGGSGGGGSGGGGS |
| 32 | SGSG Linker | SGSG |
| 33 | S(GSG)₂ Linker | SGSGGSG |
| 34 | S(GSG)₃ Linker | SGSGGSGGSG |
| 35 | S(GSG)₄ Linker | SGSGGSGGSGGSG |
| 36 | SG3S Linker | SGGGS |
| 37 | S(G3S)₂ Linker | SGGGSGGGS |
| 38 | S(G3S)₃ Linker | SGGGSGGGSGGGS |
| 39 | S(G3S)₄ Linker | SGGGSGGGSGGGSGGGS |
| 40 | SG4S Linker | SGGGGS |
| 41 | S(G4S)₂ Linker | SGGGGSGGGGS |
| 42 | S(G4S)₃ Linker | SGGGGSGGGGSGGGGS |
| 43 | S(G4S)₄ Linker | SGGGGSGGGGSGGGGSGGGG |
| | | S |
| 44 | EAAAK Linker | EAAAK |
| 45 | (EAAAK)₂ Linker | EAAAKEAAAK |
| 46 | (EAAAK)₃ Linker | EAAAKEAAAKEAAAK |
| 47 | (EAAAK)₄ Linker | EAAAKEAAAKEAAAKEAAAK |
| 48 | PAPAP Linker | PAPAP |
| 49 | (PAPAP)₂ Linker | PAPAPPAPAP |
| 50 | (PAPAP)₃ Linker | PAPAPPAPAPPAPAP |
| 51 | (PAPAP)₄ Linker | PAPAPPAPAPPAPAPPAPAP |
| 52 | VLVH Linker | IKRTVAAP |
| 53 | RAKPS Linker | RAKPS |
| 54 | (RAKPS)₂ Linker | RAKPSRAKPS |
| 55 | ASTKG Linker | ASTKG |
| 56 | (ASTKG)₂ Linker | ASTKGASTKG |
| 57 | AKTHT Linker | AKTHT |
| 58 | (AKTHT)₂ Linker | AKTHTAKTHT |
| 59 | ASTKG(G4S)₂ Linker | ASTKGGGGGSGGGGS |
| 60 | AKTHT(G4S)₂ Linker | AKTHTGGGGSGGGGS |
| 61 | RAKPS(G4S)₂ Linker | RAKPSGGGGSGGGGS |
| 62 | hIL-2 fragment | NNYKNPKLTRMLTFKFYMPK |
| | (N29 to A50) | KA |
| 63 | IL-15 hinge | NNYENPKLTDATLYMPKKA |
| | fragment- | |
| | containing | |
| | peptide 1 | |
| 64 | IL-15 hinge | QSMEIDATTFKFYMPKKA |
| | fragment- | |
| | containing | |
| | peptide 2 | |
| 65 | IL-15 hinge | QSMEIDATKFYMPKKA |
| | fragment- | |
| | containing | |
| | peptide 3 | |
| 66 | IL-15 hinge | QSMEIDATLFYMPKKA |
| | fragment- | |
| | containing | |
| | peptide 4 | |
| 67 | IL-15 hinge | QSMEIDATLYTESDV |
| | fragment- | |
| | containing | |
| | peptide 5 | |
| 68 | IL-15 hinge | NNYHNPKLTDATLYMPKKA |
| | fragment- | |
| | containing | |
| | peptide 6 | |
| 69 | IL-15 hinge | QSMHIDATTFKFYMPKKA |
| | fragment- | |
| | containing | |
| | peptide 7 | |
| 70 | IL-15 hinge | QSMHIDATKFYMPKKA |
| | fragment- | |
| | containing | |
| | peptide 8 | |
| 71 | IL-15 hinge | QSMHIDATLFYMPKKA |
| | fragment- | |
| | containing | |
| | peptide 9 | |
| 72 | IL-15 hinge | QSMHIDATLYTESDV |
| | fragment- | |
| | containing | |
| | peptide 10 | |
| 73 | IL-2-IL-15 | APTSSSTKKTQLQLEHLLLD |
| | chimera 1 | LQMILNGINNYENPKLTDAT |
| | (C125S) | LYMPKKATELKHLQCLEEEL |
| | | KPLEEVLNLAQSKNFHLRPR |
| | | DLISNINVIVLELKGSETTF |
| | | MCEYADETATIVEFLNRWIT |
| | | FSQSIISTLT |
| 74 | IL-2-IL-15 | APTSSSTKKTQLQLEHLLLD |
| | chimera 2 | LQMILNGIQSMEIDATTFKF |
| | (C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 75 | IL-2-IL-15 | APTSSSTKKTQLQLEHLLLD |
| | chimera 3 | LQMILNGIQSMEIDATKFYM |
| | (C125S) | PKKATELKHLQCLEEELKPL |
| | | EEVLNLAQSKNFHLRPRDLI |
| | | SNINVIVLELKGSETTFMCE |
| | | YADETATIVEFLNRWITFSQ |
| | | SIISTLT |
| 76 | IL-2-IL-15 | APTSSSTKKTQLQLEHLLLD |
| | chimera 4 | LQMILNGIQSMEIDATLFYM |
| | (C125S) | PKKATELKHLQCLEEELKPL |
| | | EEVLNLAQSKNFHLRPRDLI |
| | | SNINVIVLELKGSETTFMCE |
| | | YADETATIVEFLNRWITFSQ |
| | | SIISTLT |
| 77 | IL-2-IL-15 | APTSSSTKKTQLQLEHLLLD |
| | chimera 5 | LQMILNGIQSMEIDATLYTE |
| | (C125S) | SDVTELKHLQCLEEELKPLE |
| | | EVLNLAQSKNFHLRPRDLIS |
| | | NINVIVLELKGSETTFMCEY |
| | | ADETATIVEFLNRWITFSQS |
| | | IISTLT |
| 78 | IL-2-IL-15 | APTSSSTKKTQLQLEHLLLD |
| | chimera 6 | LQMILNGIQSMHIDATTFKF |
| | (C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 79 | IL-2-IL-15 | APTSSSTKKTQLQLKHLLLD |
| | chimera 7 | LQMILNGIQSMEIDATTFKF |
| | (E15K, C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 80 | IL-2-IL-15 | APTSSSTKKTQLQLEHLLLD |
| | chimera 8 | LQMILNGIQSMEIDATTFKF |
| | (S87D, C125S) | YMPKKATELKHLQCLEEELK |
| | | PLEEVLNLAQSKNFHLRPRD |
| | | LIDNINVIVLELKGSETTFM |
| | | CEYADETATIVEFLNRWITF |
| | | SQSIISTLT |
| 81 | IL-2-IL-15 | APTSSSTKKTQLQLEHLLLD |
| | chimera 9 | LQMILNGIQSMEIDATTFKF |
| | (S87E, C125S) | YMPKKATELKHLQCLEEELK |
| | | PLEEVLNLAQSKNFHLRPRD |
| | | LIENINVIVLELKGSETTFM |
| | | CEYADETATIVEFLNRWITF |
| | | SQSIISTLT |
| 82 | IL-2-IL-15 | APTSSSTKKTQLQLKHLLLD |
| | chimera 10 | LQMILNGIQSMEIDATTFKF |
| | (E15K, S87D, C125S) | YMPKKATELKHLQCLEEELK |
| | | PLEEVLNLAQSKNFHLRPRD |
| | | LIDNINVIVLELKGSETTFM |
| | | CEYADETATIVEFLNRWITF |
| | | SQSIISTLT |
| 83 | IL-2-IL-15 | APTSSSTKKTQLQLKHLLLD |
| | chimera 11 | LQMILNGIQSMEIDATTFKF |
| | (E15K, S87E, C125S) | YMPKKATELKHLQCLEEELK |
| | | PLEEVLNLAQSKNFHLRPRD |
| | | LIENINVIVLELKGSETTFM |
| | | CEYADETATIVEFLNRWITF |
| | | SQSIISTLT |
| 84 | IL-2-IL-15 | APTSSSTKKTQLQLKHLLLD |
| | chimera 12 | LQMILNGIQSMHIDATTFKF |
| | (E15K, C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 85 | IL-2-IL-15 | APTSSSTKKTQLQLEHLLLD |
| | chimera 13 | LQMILNGIQSMHIDATTFKF |
| | (S87D, C125S) | YMPKKATELKHLQCLEEELK |
| | | PLEEVLNLAQSKNFHLRPRD |
| | | LIDNINVIVLELKGSETTFM |
| | | CEYADETATIVEFLNRWITF |
| | | SQSIISTLT |
| 86 | IL-2-IL-15 | APTSSSTKKTQLQLEHLLLD |
| | chimera 14 | LQMILNGIQSMHIDATTFKF |
| | (S87E, C125S) | YMPKKATELKHLQCLEEELK |
| | | PLEEVLNLAQSKNFHLRPRD |
| | | LIENINVIVLELKGSETTFM |
| | | CEYADETATIVEFLNRWITF |
| | | SQSIISTLT |
| 87 | IL-2-IL-15 | APTSSSTKKTQLQLKHLLLD |
| | chimera 15 | LQMILNGIQSMHIDATTFKF |
| | (E15K, S87D, C125S) | YMPKKATELKHLQCLEEELK |
| | | PLEEVLNLAQSKNFHLRPRD |
| | | LIDNINVIVLELKGSETTFM |
| | | CEYADETATIVEFLNRWITF |
| | | SQSIISTLT |
| 88 | IL-2-IL-15 | APTSSSTKKTQLQLKHLLLD |
| | chimera 16 | LQMILNGIQSMHIDATTFKF |
| | (E15K, S87E, C125S) | YMPKKATELKHLQCLEEELK |
| | | PLEEVLNLAQSKNFHLRPRD |
| | | LIENINVIVLELKGSETTFM |
| | | CEYADETATIVEFLNRWITF |
| | | SQSIISTLT |
| 89 | IL-2-IL-15 | APASSSTKKTQLQLEHLLLD |
| | chimera 17 | LQMILNGINNYENPKLTDAT |
| | (T3A, C125S) | LYMPKKATELKHLQCLEEEL |
| | | KPLEEVLNLAQSKNFHLRPR |
| | | DLISNINVIVLELKGSETTF |
| | | MCEYADETATIVEFLNRWIT |
| | | FSQSIISTLT |
| 90 | IL-2-IL-15 | APASSSTKKTQLQLEHLLLD |
| | chimera 18 | LQMILNGIQSMEIDATTFKF |
| | (T3A, C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 91 | IL-2-IL-15 | APASSSTKKTQLQLEHLLLD |
| | chimera 19 | LQMILNGIQSMEIDATKFYM |
| | (T3A, C125S) | PKKATELKHLQCLEEELKPL |
| | | EEVLNLAQSKNFHLRPRDLI |
| | | SNINVIVLELKGSETTFMCE |
| | | YADETATIVEFLNRWITFSQ |
| | | SIISTLT |
| 92 | IL-2-IL-15 | APASSSTKKTQLQLEHLLLD |
| | chimera 20 | LQMILNGIQSMEIDATLFYM |
| | (T3A, C125S) | PKKATELKHLQCLEEELKPL |
| | | EEVLNLAQSKNFHLRPRDLI |
| | | SNINVIVLELKGSETTFMCE |
| | | YADETATIVEFLNRWITFSQ |
| | | SIISTLT |
| 93 | IL-2-IL-15 | APASSSTKKTQLQLEHLLLD |
| | chimera 21 | LQMILNGIQSMEIDATLYTE |
| | (T3A, C125S) | SDVTELKHLQCLEEELKPLE |
| | | EVLNLAQSKNFHLRPRDLIS |
| | | NINVIVLELKGSETTFMCEY |
| | | ADETATIVEFLNRWITFSQS |
| | | IISTLT |
| 94 | IL-2-IL-15 | APASSSTKKTQLQLEHLLLD |
| | chimera 22 | LQMILNGIQSMHIDATTFKF |
| | (T3A, C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 95 | IL-2-IL-15 | APASSSTKKTQLQLKHLLLD |
| | chimera 23 | LQMILNGIQSMEIDATTFKF |
| | (T3A, E15K, C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 96 | IL-2-IL-15 | APASSSTKKTQLQLEHLLLD |
| | chimera 24 | LQMILNGIQSMEIDATTFKF |
| | (T3A, S87D, C125S) | YMPKKATELKHLQCLEEELK |
| | | PLEEVLNLAQSKNFHLRPRD |
| | | LIDNINVIVLELKGSETTFM |
| | | CEYADETATIVEFLNRWITF |
| | | SQSIISTLT |
| 97 | IL-2-IL-15 | APASSSTKKTQLQLEHLLLD |
| | chimera 25 | LQMILNGIQSMEIDATTFKF |
| | (T3A, S87E, C125S) | YMPKKATELKHLQCLEEELK |
| | | PLEEVLNLAQSKNFHLRPRD |
| | | LIENINVIVLELKGSETTFM |
| | | CEYADETATIVEFLNRWITF |
| | | SQSIISTLT |
| 98 | IL-2-IL-15 | APASSSTKKTQLQLKHLLLD |
| | chimera 26 | LQMILNGIQSMEIDATTFKF |
| | (T3A, E15K, | YMPKKATELKHLQCLEEELK |
| | S87D, C125S) | PLEEVLNLAQSKNFHLRPRD |
| | | LIDNINVIVLELKGSETTFM |
| | | CEYADETATIVEFLNRWITF |
| | | SQSIISTLT |
| 99 | IL-2-IL-15 | APASSSTKKTQLQLKHLLLD |
| | chimera 27 | LQMILNGIQSMEIDATTFKF |
| | (T3A, E15K, | YMPKKATELKHLQCLEEELK |
| | S87E, C125S) | PLEEVLNLAQSKNFHLRPRD |
| | | LIENINVIVLELKGSETTFM |
| | | CEYADETATIVEFLNRWITF |
| | | SQSIISTLT |
| 100 | IL-2-IL-15 | APASSSTKKTQLQLKHLLLD |
| | chimera 28 | LQMILNGIQSMHIDATTFKF |
| | (T3A, E15K, C125S) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | SQSIISTLT |
| 101 | IL-2-IL-15 | APASSSTKKTQLQLEHLLLD |
| | chimera 29 | LQMILNGIQSMHIDATTFKF |
| | (T3A, S87D, C125S) | YMPKKATELKHLQCLEEELK |
| | | PLEEVLNLAQSKNFHLRPRD |
| | | LIDNINVIVLELKGSETTFM |
| | | CEYADETATIVEFLNRWITF |
| | | SQSIISTLT |
| 102 | IL-2-IL-15 | APASSSTKKTQLQLEHLLLD |
| | chimera 30 | LQMILNGIQSMHIDATTFKF |
| | (T3A, S87E, C125S) | YMPKKATELKHLQCLEEELK |
| | | PLEEVLNLAQSKNFHLRPRD |
| | | LIENINVIVLELKGSETTFM |
| | | CEYADETATIVEFLNRWITF |
| | | SQSIISTLT |
| 103 | IL-2-IL-15 | APASSSTKKTQLQLKHLLLD |
| | chimera 31 | LQMILNGIQSMHIDATTFKF |
| | (T3A, E15K, | YMPKKATELKHLQCLEEELK |
| | S87D, C125S) | PLEEVLNLAQSKNFHLRPRD |
| | | LIDNINVIVLELKGSETTFM |
| | | CEYADETATIVEFLNRWITF |
| | | SQSIISTLT |
| 104 | IL-2-IL-15 | APASSSTKKTQLQLKHLLLD |
| | chimera 32 | LQMILNGIQSMHIDATTFKF |
| | (T3A, E15K, | YMPKKATELKHLQCLEEELK |
| | S87E, C125S) | PLEEVLNLAQSKNFHLRPRD |
| | | LIENINVIVLELKGSETTFM |
| | | CEYADETATIVEFLNRWITF |
| | | SQSIISTLT |
| 105 | hIL-2 mutein- | APTSSSTKKTQLQLEHLLLD |
| | anti-HSA | LQMILNGINNYKNPKLTRML |
| | (C125S) C1 | TFKFYMPKKATELKHLQCLE |
| | | EELKPLEEVLNLAQSKNFHL |
| | | RPRDLISNINVIVLELKGSE |
| | | TTFMCEYADETATIVEFLNR |
| | | WITFSQSIISTLTGGGGSGG |
| | | GGSGGGGSGGGGSEVQLVES |
| | | GGGLVQPGGSLRLSCAASG |
| | | STWSINTLAWYRQAPGKQRD |
| | | LVARISSGGSTYYADSVKGR |
| | | FTISRDNSKNTLYLQMNSLR |
| | | AEDTAVYYCYAQSTWYPPSW |
| | | GQGTLVTVSS |
| 106 | Anti-HSA-IL-2 A1 | QVQLVESGGGVVQPGGSLRL |
| | (IL-2-IL15 | SCAASGFAFRGFGMSWVRQA |
| | chimera 7) | PGKGLEWVSSINNGGSDTYY |
| | | ADSVKGRFTISRDNSKNTLY |
| | | LQMNSLRAEDTAVYYCAIGG |
| | | PGASPSGQGTQVTVSSGGGG |
| | | SGGGGSGGGGSAPTSSSTKK |
| | | TQLQLKHLLLDLQMILNGIQ |
| | | SMEIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 107 | Anti-HSA-IL-2 A2 | QVQLVESGGGVVQPGGSLRL |
| | (IL-2-IL15 | SCAASGFAFRGFGMSWVRQA |
| | chimera 12) | PGKGLEWVSSINNGGSDTYY |
| | | ADSVKGRFTISRDNSKNTLY |
| | | LQMNSLRAEDTAVYYCAIGG |
| | | PGASPSGQGTQVTVSSGGGG |
| | | SGGGGSGGGGSAPTSSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 108 | Anti-HSA-IL-2 A3 | EVOLVESGGGLVQPGGSLRL |
| | (IL-2-IL15 | SCAASGSTWSINTLAWYRQA |
| | chimera 7) | PGKQRDLVARISSGGSTYYA |
| | | DSVKGRFTISRDNSKNTLYL |
| | | QMNSLRAEDTAVYYCYAQST |
| | | WYPPSWGQGTLVTVSSGGGG |
| | | SGGGGSGGGGSAPTSSSTKK |
| | | TQLQLKHLLLDLQMILNGIQ |
| | | SMEIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 109 | Anti-HSA-IL-2 A4 | EVOLVESGGGLVQPGGSLRL |
| | (IL-2-IL15 | SCAASGSTWSINTLAWYRQA |
| | chimera 12) | PGKQRDLVARISSGGSTYYA |
| | | DSVKGRFTISRDNSKNTLYL |
| | | QMNSLRAEDTAVYYCYAQST |
| | | WYPPSWGQGTLVTVSSGGGG |
| | | SGGGGSGGGGSAPTSSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 110 | Anti-HSA-IL-2 A5 | EVOLVESGGGLVQPGGSLRL |
| | (IL-2-IL15 | SCAASGSTWSINTLAWYRQA |
| | chimera 15) | PGKQRDLVARISSGGSTYYA |
| | | DSVKGRFTISRDNSKNTLYL |
| | | QMNSLRAEDTAVYYCYAQST |
| | | WYPPSWGQGTLVTVSSRAKP |
| | | SGGGGSGGGGSAPTSSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLIDNINVIV |
| | | LELKGSETTFMCEYADETAT |
| | | IVEFLNRWITFSQSIISTLT |
| 111 | Anti-HSA-IL-2 A6 | EVOLVESGGGLVQPGGSLRL |
| | (IL-2-IL15 | SCAASGSTWSINTLAWYRQA |
| | chimera 15) | PGKQRDLVARISSGGSTYYA |
| | | DSVKGRFTISRDNSKNTLYL |
| | | QMNSLRAEDTAVYYCYAQST |
| | | WYPPSWGQGTLVTVSSAKTH |
| | | TGGGGSGGGGSSAPTSSSTK |
| | | KTQLQLKHLLLDLQMILNGI |
| | | QSMHIDATTFKFYMPKKATE |
| | | LKHLQCLEEELKPLEEVLNL |
| | | AQSKNFHLRPRDLIDNINVI |
| | | VLELKGSETTFMCEYADETA |
| | | TIVEFLNRWITFSQSIISTL |
| | | T |
| 112 | Anti-HSA-IL-2 A7 | QVQLVESGGGVVQPGGSLRL |
| | (IL-2-IL15 | SCAASGFAFRGFGMSWVRQA |
| | chimera 23) | PGKGLEWVSSINNGGSDTYY |
| | | ADSVKGRFTISRDNSKNTLY |
| | | LQMNSLRAEDTAVYYCAIGG |
| | | PGASPSGQGTQVTVSSGGGG |
| | | SGGGGSGGGGSAPASSSTKK |
| | | TQLQLKHLLLDLQMILNGIQ |
| | | SMEIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 113 | Anti-HSA-IL-2 A8 | QVQLVESGGGVVQPGGSLRL |
| | (IL-2-IL15 | SCAASGFAFRGFGMSWVRQA |
| | chimera 28) | PGKGLEWVSSINNGGSDTYY |
| | | ADSVKGRFTISRDNSKNTLY |
| | | LQMNSLRAEDTAVYYCAIGG |
| | | PGASPSGQGTQVTVSSGGGG |
| | | SGGGGSGGGGSAPASSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 114 | Anti-HSA-IL-2 A9 | EVOLVESGGGLVQPGGSLRL |
| | (IL-2-IL15 | SCAASGSTWSINTLAWYRQA |
| | chimera 23) | PGKQRDLVARISSGGSTYYA |
| | | DSVKGRFTISRDNSKNTLYL |
| | | QMNSLRAEDTAVYYCYAQST |
| | | WYPPSWGQGTLVTVSSGGGG |
| | | SGGGGSGGGGSAPASSSTKK |
| | | TQLQLKHLLLDLQMILNGIQ |
| | | SMEIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 115 | Anti-HSA-IL-2 A10 | EVOLVESGGGLVQPGGSLRL |
| | (IL-2-IL15 | SCAASGSTWSINTLAWYRQA |
| | chimera 28) | PGKQRDLVARISSGGSTYYA |
| | | DSVKGRFTISRDNSKNTLYL |
| | | QMNSLRAEDTAVYYCYAQST |
| | | WYPPSWGQGTLVTVSSGGGG |
| | | SGGGGSGGGGSAPASSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 116 | Anti-HSA-IL-2 A11 | EVOLVESGGGLVQPGGSLRL |
| | (IL-2-IL15 | SCAASGSTWSINTLAWYRQA |
| | chimera 31) | PGKQRDLVARISSGGSTYYA |
| | | DSVKGRFTISRDNSKNTLYL |
| | | QMNSLRAEDTAVYYCYAQST |
| | | WYPPSWGQGTLVTVSSRAKP |
| | | SGGGGSGGGGSAPASSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLIDNINVIV |
| | | LELKGSETTFMCEYADETAT |
| | | IVEFLNRWITFSQSIISTLT |
| 117 | Anti-HSA-IL-2 A12 | EVOLVESGGGLVQPGGSLRL |
| | (IL-2-IL15 | SCAASGSTWSINTLAWYRQA |
| | chimera 31) | PGKQRDLVARISSGGSTYYA |
| | | DSVKGRFTISRDNSKNTLYL |
| | | QMNSLRAEDTAVYYCYAQST |
| | | WYPPSWGQGTLVTVSSAKTH |
| | | TGGGGSGGGGSSAPASSSTK |
| | | KTQLQLKHLLLDLQMILNGI |
| | | QSMHIDATTFKFYMPKKATE |
| | | LKHLQCLEEELKPLEEVLNL |
| | | AQSKNFHLRPRDLIDNINVI |
| | | VLELKGSETTFMCEYADETA |
| | | TIVEFLNRWITFSQSIISTL |
| | | T |
| 118 | IL-2-IL-15 | APTSSSTKKTQLQLEHLCLD |
| | chimera 33 | LQMILNGIQSMEIDATTFKF |
| | (L18C, Q126E) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | CESIISTLT |
| 119 | IL-2-IL-15 | APASSSTKKTQLQLEHLCLD |
| | chimera 34 | LQMILNGIQSMEIDATTFKF |
| | (T3A, L18C, Q126E) | YMPKKATELKHLQCLEEELK |
| | | PLEEVLNLAQSKNFHLRPRD |
| | | LISNINVIVLELKGSETTFM |
| | | CEYADETATIVEFLNRWITF |
| | | CESIISTLT |
| 120 | Anti-HSA-IL-2 A13 | EVOLVESGGGLVQPGGSLRL |
| | (IL-2-IL15 | SCAASGSTWSINTLAWYRQA |
| | chimera 33) | PGKQRDLVARISSGGSTYYA |
| | | DSVKGRFTISRDNSKNTLYL |
| | | QMNSLRAEDTAVYYCYAQST |
| | | WYPPSWGQGTLVTVSSGGGG |
| | | SGGGGSGGGGSAPTSSSTKK |
| | | TQLQLEHLCLDLQMILNGIQ |
| | | SMEIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFCESIISTLT |
| 121 | Anti-HSA-IL-2 A14 | EVOLVESGGGLVQPGGSLRL |
| | (IL-2-IL15 | SCAASGSTWSINTLAWYRQA |
| | chimera 34) | PGKQRDLVARISSGGSTYYA |
| | | DSVKGRFTISRDNSKNTLYL |
| | | QMNSLRAEDTAVYYCYAQST |
| | | WYPPSWGQGTLVTVSSGGGG |
| | | SGGGGSGGGGSAPASSSTKK |
| | | TQLQLEHLCLDLQMILNGIQ |
| | | SMEIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFCESIISTLT |
| 122 | Anti-HSA-IL-2 A15 | QVQLVESGGGVVQPGGSLRL |
| | (IL-2-IL15 | SCAASGFAFRGFGMSWVRQA |
| | chimera 12) | PGKGLEWVSSINNGGSDTYY |
| | | ADSVKGRFTISRDNSKNTLY |
| | | LQMNSLRAEDTAVYYCAIGG |
| | | PGASPSGQGTQVTVSSASTK |
| | | GGGGGSGGGGSAPTSSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 123 | Anti-HSA-IL-2 A16 | QVQLVESGGGVVQPGGSLRL |
| | (IL-2-IL15 | SCAASGFAFRGFGMSWVRQA |
| | chimera 28) | PGKGLEWVSSINNGGSDTYY |
| | | ADSVKGRFTISRDNSKNTLY |
| | | LQMNSLRAEDTAVYYCAIGG |
| | | PGASPSGQGTQVTVSSASTK |
| | | GGGGGSGGGGSAPASSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 124 | Anti-HSA-IL-2 A17 | EVOLVESGGGLVQPGGSLRL |
| | (IL-2-IL15 | SCAASGSTWSINTLAWYRQA |
| | chimera 12) | PGKQRDLVARISSGGSTYYA |
| | | DSVKGRFTISRDNSKNTLYL |
| | | QMNSLRAEDTAVYYCYAQST |
| | | WYPPSWGQGTLVTVSSASTK |
| | | GGGGGSGGGGSAPTSSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |
| 125 | Anti-HSA-IL-2 A18 | EVOLVESGGGLVQPGGSLRL |
| | (IL-2-IL15 | SCAASGSTWSINTLAWYRQA |
| | chimera 28) | PGKQRDLVARISSGGSTYYA |
| | | DSVKGRFTISRDNSKNTLYL |
| | | QMNSLRAEDTAVYYCYAQST |
| | | WYPPSWGQGTLVTVSSASTK |
| | | GGGGGSGGGGSAPASSSTKK |
| | | TQLQLKHLLLDLQMILNGIQ |
| | | SMHIDATTFKFYMPKKATEL |
| | | KHLQCLEEELKPLEEVLNLA |
| | | QSKNFHLRPRDLISNINVIV |
| | | LELKGSETTFMCEYADETAT |
| | | IVEFLNRWITFSQSIISTLT |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A fusion protein comprising an IL-2 domain, an anti-HSA sdAb, and optionally a peptide linker; wherein the C-terminus of the anti-HSA sdAb is connected to the N-terminus of the IL-2 domain directly or via the peptide linker; and wherein the IL-2 domain of SEQ ID NO: 1 comprises (i) a replacement of the amino acid residues from positions 29 to 50 as set forth in SEQ ID NO: 1 with a peptide comprising the amino acid sequence of an IL-15 hinge or a fragment thereof and (ii) an amino acid substitution.

2. The fusion protein of claim 1, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution at position T3, K8, K9, Q13, E15, H16, L18, L19, M23, K32, K76, H79, R81, D84, S87, N88, V91, I92, E95, C125, Q126, S127, or S130 as set forth in SEQ ID NO: 1.

3. The fusion protein of claim 1 or 2, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution at position T3, K8, K9, E15, H16, L18, K32, K76, H79, R81, D84, S87, N88, 192, E95, C125, Q126, S127, or S130 as set forth in SEQ ID NO: 1.

4. The fusion protein of any one of claims 1 to 3, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution at position T3, E15, L18, K32, K76, S87, C125, or Q126 as set forth in SEQ ID NO: 1.

5. The fusion protein of any one of claims 1 to 4, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution of T3A, E15K, E15Q, L18C, K32D, K32E, K32Q, K76D, K76E, K76Q, S87D, S87E, C125A, C125S, or Q126E.

6. The fusion protein of any one of claims 1 to 4, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution at position T3, E15, L18, S87, C125, or Q126 as set forth in SEQ ID NO: 1.

7. The fusion protein of any one of claims 1 to 6, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution of T3A, E15K, E15Q, L18C, S87D, S87E, C125A, or C125S.

8. The fusion protein of any one of claims 1 to 4 and 6, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution at position T3, E15, S87, or C125 as set forth in SEQ ID NO: 1.

9. The fusion protein of any one of claims 1 to 8, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution of T3A, E15K, S87D, S87E, or C125S.

10. The fusion protein of any one of claims 1 to 4, 6, and 8, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) two amino acid substitutions, each independently at position T3, E15, S87, or C125 as set forth in SEQ ID NO: 1.

11. The fusion protein of any one of claims 1 to 10, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) two amino acid substitutions, each independently selected from T3A, E15K, S87D, S87E, and C125S.

12. The fusion protein of any one of claims 1 to 11, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof, and (ii) two amino acid substitutions of E15K, and S87D or S87E.

13. The fusion protein of any one of claims 1 to 11, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) two amino acid substitutions of E15K and C125S.

14. The fusion protein of any one of claims 1 to 11, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof, and (ii) two amino acid substitutions of S87D or S87E, and C125S.

15. The fusion protein of any one of claims 1 to 4, 6, 8, and 10, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) three amino acid substitutions, each independently at position T3, E15, S87, or C125 as set forth in SEQ ID NO: 1.

16. The fusion protein of any one of claims 1 to 15, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) three amino acid substitutions, each independently selected from T3A, E15K, S87D, S87E, and C125S.

17. The fusion protein of any one of claims 1 to 16, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) three amino acid substitutions of E15K, S87D or S87E, and C125S.

18. The fusion protein of any one of claims 1 to 4, 6, 8, 10, and 15, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) four amino acid substitutions at position T3, E15, S87, and C125 as set forth in SEQ ID NO: 1.

19. The fusion protein of any one of claims 1 to 18, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) four amino acid substitutions of T3A, E15K, S87D or S87E, and C125S.

20. The fusion protein of any one of claims 1 to 4 and 6, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution at position L18 and an amino acid substitution at T3 or Q126 as set forth in SEQ ID NO: 1.

21. The fusion protein of any one of claims 1 to 6 and 20, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) an amino acid substitution of L18C and an amino acid substitution of T3A or Q126E.

22. The fusion protein of any one of claims 1 to 6, 20, and 21, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof; and (ii) amino acid substitutions of L18C and Q126E.

23. The fusion protein of any one of claims 1 to 6 and 20 to 22, wherein the IL-2 domain comprises (i) a replacement of the amino acid residues from positions 29 to 50 as set forth in SEQ ID NO: 1 with a peptide comprising the amino acid sequence of an IL-15 hinge or a fragment thereof, and (ii) amino acid substitutions of T3A. L18C, and Q126E.

24. The fusion protein of any one of claims 1 to 23, wherein the peptide comprising the amino acid sequence of an IL-15 hinge or a fragment thereof comprises an amino acid sequence of SEQ ID NOs: 63 to 72.

25. The fusion protein of any one of claims 1 to 24, wherein the peptide comprising the amino acid sequence of an IL-15 hinge or a fragment thereof comprises an amino acid sequence of SEQ ID NO: 63.

26. The fusion protein of any one of claims 1 to 24, wherein the peptide comprising the amino acid sequence of an IL-15 hinge or a fragment thereof comprises an amino acid sequence of SEQ ID NO: 64.

27. The fusion protein of any one of claims 1 to 24, wherein the peptide comprising the amino acid sequence of an IL-15 hinge or a fragment thereof comprises an amino acid sequence of SEQ ID NO: 65.

28. The fusion protein of any one of claims 1 to 24, wherein the peptide comprising the amino acid sequence of an IL-15 hinge or a fragment thereof comprises an amino acid sequence of SEQ ID NO: 66.

29. The fusion protein of any one of claims 1 to 24, wherein the peptide comprising the amino acid sequence of an IL-15 hinge or a fragment thereof comprises an amino acid sequence of SEQ ID NO: 67.

30. The fusion protein of any one of claims 1 to 24, wherein the peptide comprising the amino acid sequence of an IL-15 hinge or a fragment thereof comprises an amino acid sequence of SEQ ID NO: 68.

31. The fusion protein of any one of claims 1 to 24, wherein the peptide comprising the amino acid sequence of an IL-15 hinge or a fragment thereof comprises an amino acid sequence of SEQ ID NO: 69.

32. The fusion protein of any one of claims 1 to 24, wherein the peptide comprising the amino acid sequence of an IL-15 hinge or a fragment thereof comprises an amino acid sequence of SEQ ID NO: 70.

33. The fusion protein of any one of claims 1 to 24, wherein the peptide comprising the amino acid sequence of an IL-15 hinge or a fragment thereof comprises an amino acid sequence of SEQ ID NO: 71.

34. The fusion protein of any one of claims 1 to 24, wherein the peptide comprising the amino acid sequence of an IL-15 hinge or a fragment thereof comprises an amino acid sequence of SEQ ID NO: 72.

35. The fusion protein of any one of claims 1 to 34, wherein the IL-2 domain comprises the amino acid sequence of any one of SEQ ID NOs: 73 to 104, 118, and 119.

36. The fusion protein of any one of claims 1 to 35, wherein the IL-2 domain has the amino acid sequence of SEQ ID NO: 73.

37. The fusion protein of any one of claims 1 to 35, wherein the IL-2 domain has the amino acid sequence of SEQ ID NO: 74.

38. The fusion protein of any one of claims 1 to 35, wherein the IL-2 domain has the amino acid sequence of SEQ ID NO: 75.

39. The fusion protein of any one of claims 1 to 35, wherein the IL-2 domain has the amino acid sequence of SEQ ID NO: 76.

40. The fusion protein of any one of claims 1 to 35, wherein the IL-2 domain has the amino acid sequence of SEQ ID NO: 77.

41. The fusion protein of any one of claims 1 to 35, wherein the IL-2 domain has the amino acid sequence of SEQ ID NO: 78.

42. The fusion protein of any one of claims 1 to 35, wherein the IL-2 domain has the amino acid sequence of SEQ ID NO: 79.

43. The fusion protein of any one of claims 1 to 35, wherein the IL-2 domain has the amino acid sequence of SEQ ID NO: 80.

44. The fusion protein of any one of claims 1 to 35, wherein the IL-2 domain has the amino acid sequence of SEQ ID NO: 81.

45. The fusion protein of any one of claims 1 to 35, wherein the IL-2 domain has the amino acid sequence of SEQ ID NO: 82.

46. The fusion protein of any one of claims 1 to 35, wherein the IL-2 domain has the amino acid sequence of SEQ ID NO: 83.

47. The fusion protein of any one of claims 1 to 35, wherein the IL-2 domain has the amino acid sequence of SEQ ID NO: 84.

48. The fusion protein of any one of claims 1 to 35, wherein the IL-2 domain has the amino acid sequence of SEQ ID NO: 85.

49. The fusion protein of any one of claims 1 to 35, wherein the IL-2 domain has the amino acid sequence of SEQ ID NO: 86.

50. The fusion protein of any one of claims 1 to 35, wherein the IL-2 domain has the amino acid sequence of SEQ ID NO: 87.

51. The fusion protein of any one of claims 1 to 35, wherein the IL-2 domain has the amino acid sequence of SEQ ID NO: 88.

52. The fusion protein of any one of claims 1 to 35, wherein the IL-2 domain has the amino acid sequence of SEQ ID NO: 89.

53. The fusion protein of any one of claims 1 to 35, wherein the IL-2 domain has the amino acid sequence of SEQ ID NO: 90.

54. The fusion protein of any one of claims 1 to 35, wherein the IL-2 domain has the amino acid sequence of SEQ ID NO: 91.

55. The fusion protein of any one of claims 1 to 35, wherein the IL-2 domain has the amino acid sequence of SEQ ID NO: 92.

56. The fusion protein of any one of claims 1 to 35, wherein the IL-2 domain has the amino acid sequence of SEQ ID NO: 93.

57. The fusion protein of any one of claims 1 to 35, wherein the IL-2 domain has the amino acid sequence of SEQ ID NO: 94.

58. The fusion protein of any one of claims 1 to 35, wherein the IL-2 domain has the amino acid sequence of SEQ ID NO: 95.

59. The fusion protein of any one of claims 1 to 35, wherein the IL-2 domain has the amino acid sequence of SEQ ID NO: 96.

60. The fusion protein of any one of claims 1 to 35, wherein the IL-2 domain has the amino acid sequence of SEQ ID NO: 97.

61. The fusion protein of any one of claims 1 to 35, wherein the IL-2 domain has the amino acid sequence of SEQ ID NO: 98.

62. The fusion protein of any one of claims 1 to 35, wherein the IL-2 domain has the amino acid sequence of SEQ ID NO: 99.

63. The fusion protein of any one of claims 1 to 35, wherein the IL-2 domain has the amino acid sequence of SEQ ID NO: 100.

64. The fusion protein of any one of claims 1 to 35, wherein the IL-2 domain has the amino acid sequence of SEQ ID NO: 101.

65. The fusion protein of any one of claims 1 to 35, wherein the IL-2 domain has the amino acid sequence of SEQ ID NO: 102.

66. The fusion protein of any one of claims 1 to 35, wherein the IL-2 domain has the amino acid sequence of SEQ ID NO: 103.

67. The fusion protein of any one of claims 1 to 35, wherein the IL-2 domain has the amino acid sequence of SEQ ID NO: 104.

68. The fusion protein of any one of claims 1 to 35, wherein the IL-2 domain has the amino acid sequence of SEQ ID NO: 118.

69. The fusion protein of any one of claims 1 to 35, wherein the IL-2 domain has the amino acid sequence of SEQ ID NO: 119.

70. The fusion protein of any one of claims 1 to 69, wherein the anti-HSA sdAb comprises (i) CDR1 of SEQ ID NO: 6, CDR2 of SEQ ID NO: 7, and CDR3 of SEQ ID NO: 8; or (ii) CDR1 of SEQ ID NO: 14, CDR2 of SEQ ID NO: 15, and CDR3 of SEQ ID NO: 16.

71. The fusion protein of any one of claims 1 to 70, wherein the anti-HSA sdAb comprises CDR1 of SEQ ID NO: 6, CDR2 of SEQ ID NO: 7, and CDR3 of SEQ ID NO: 8.

72. The fusion protein of any one of claims 1 to 71, wherein the single domain antibody has an amino acid sequence of SEQ ID NO: 13.

73. The fusion protein of any one of claims 1 to 70, wherein the anti-HSA sdAb comprises CDR1 of SEQ ID NO: 14, CDR2 of SEQ ID NO: 15, and CDR3 of SEQ ID NO: 16.

74. The fusion protein of any one of claims 1 to 70 and 73, wherein the single domain antibody has an amino acid sequence of SEQ ID NO: 20.

75. The fusion protein of any one of claims 1 to 74, wherein the anti-HSA sdAb is a humanized antibody.

76. The fusion protein of any one of claims 1 to 75, wherein the fusion protein comprises a peptide linker.

77. The fusion protein of any one of claims 1 to 76, wherein the peptide linker has an amino acid sequence of GSG or one of SEQ ID NOs: 21 to 61.

78. The fusion protein of any one of claims 1 to 77, wherein the peptide linker has an amino acid sequence of SEQ ID NO: 30, 59, 60, or 61.

79. The fusion protein of any one of claims 1 to 78, comprising an amino acid sequence of any one of SEQ ID NOs: 106 to 117 and 120 to 125.

80. The fusion protein of any one of claims 1 to 78, comprising an amino acid sequence of SEQ ID NO: 106.

81. The fusion protein of any one of claims 1 to 78, comprising an amino acid sequence of SEQ ID NO: 107.

82. The fusion protein of any one of claims 1 to 78, comprising an amino acid sequence of SEQ ID NO: 108.

83. The fusion protein of any one of claims 1 to 78, comprising an amino acid sequence of SEQ ID NO: 109.

84. The fusion protein of any one of claims 1 to 78, comprising an amino acid sequence of SEQ ID NO: 110.

85. The fusion protein of any one of claims 1 to 78, comprising an amino acid sequence of SEQ ID NO: 111.

86. The fusion protein of any one of claims 1 to 78, comprising an amino acid sequence of SEQ ID NO: 112.

87. The fusion protein of any one of claims 1 to 78, comprising an amino acid sequence of SEQ ID NO: 113.

88. The fusion protein of any one of claims 1 to 78, comprising an amino acid sequence of SEQ ID NO: 114.

89. The fusion protein of any one of claims 1 to 78, comprising an amino acid sequence of SEQ ID NO: 115.

90. The fusion protein of any one of claims 1 to 78, comprising an amino acid sequence of SEQ ID NO: 116.

91. The fusion protein of any one of claims 1 to 78, comprising an amino acid sequence of SEQ ID NO: 117.

92. The fusion protein of any one of claims 1 to 78, comprising an amino acid sequence of SEQ ID NO: 120.

93. The fusion protein of any one of claims 1 to 78, comprising an amino acid sequence of SEQ ID NO: 121.

94. The fusion protein of any one of claims 1 to 78, comprising an amino acid sequence of SEQ ID NO: 122.

95. The fusion protein of any one of claims 1 to 78, comprising an amino acid sequence of SEQ ID NO: 123.

96. The fusion protein of any one of claims 1 to 78, comprising an amino acid sequence of SEQ ID NO: 124.

97. The fusion protein of any one of claims 1 to 78, comprising an amino acid sequence of SEQ ID NO: 125.

98. The fusion protein of any one of claims 1 to 97, wherein the fusion protein is N-glycosylated.

99. The fusion protein of any one of claims 1 to 98, wherein the fusion protein is isolated.

100. The fusion protein of any one of claims 1 to 99, wherein the fusion protein has a K_{d} to an IL-2Rα of no less than about 1 μM.

101. A pharmaceutical composition comprising the fusion protein of any one of claims 1 to 100, and a pharmaceutically acceptable excipient.

102. The pharmaceutical composition of claim 101, wherein the pharmaceutical composition is in single dosage form.

103. The pharmaceutical composition of claim 101 or 102, wherein the pharmaceutical composition is a solid.

104. The pharmaceutical composition of claim 101 or 102, wherein the pharmaceutical composition is in a parenteral dosage form.

105. The pharmaceutical composition of claim 104, the pharmaceutical composition is in an intravenous dosage form.

106. The pharmaceutical composition of any one of claims 101, 102, 104, and 105, wherein the pharmaceutical composition is a solution.

107. A method of treating one or more symptoms of treating, preventing, or ameliorating one or more symptoms of a proliferative disease in a subject, comprising administering to the subject in need thereof a therapeutically effective amount of the fusion protein of any one of claims 1 to 100 or the pharmaceutical composition of any one of claims 101 to 106.

108. The method of claim 107, wherein the proliferative disease is cancer.

109. The method of claim 107 or 108, wherein the proliferative disease is metastatic cancer.

110. The method of any one of claims 107 to 109, wherein the proliferative disease is melanoma, ovarian cancer, pancreatic cancer, or renal cell carcinoma.

111. A method of activating an immune effector cell, comprising contacting the cell with an effective amount of the fusion protein of any one of claims 1 to 100 or the pharmaceutical composition of any one of claims 101 to 106.


SEQUENCE LISTING (125 sequences)

SEQ ID NO: 1 - protein, 133 aa, Homo sapiens [sequence omitted]

SEQ ID NO: 2 - protein, 133 aa, synthetic construct
  REGION 1..133: note = hIL-2 mutein (C125S)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 4 - protein, 213 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 5 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 6 - protein, 8 aa, synthetic construct
  REGION 1..8: note = Anti-HSA-1 CDR1
GSTWSINT

SEQ ID NO: 7 - protein, 7 aa, synthetic construct
  REGION 1..7: note = Anti-HSA-1 CDR2
ISSGGST

SEQ ID NO: 8 - protein, 10 aa, synthetic construct
  REGION 1..10: note = Anti-HSA-1 CDR3
YAQSTWYPPS

SEQ ID NO: 9 - protein, 25 aa, synthetic construct [sequence omitted]

SEQ ID NO: 10 - protein, 17 aa, synthetic construct
  REGION 1..17: note = Anti-HSA-1 FR2
LAWYRQAPGKQRDLVAR

SEQ ID NO: 11 - protein, 38 aa, synthetic construct
  REGION 1..38: note = Anti-HSA-1 FR3
YYADSVKGRFTISRDNSKNTLYLQMNSLRAEDTAVYYC

SEQ ID NO: 12 - protein, 11 aa, synthetic construct
  REGION 1..11: note = Anti-HSA-1 FR4
WGQGTLVTVSS

SEQ ID NO: 13 - protein, 116 aa, synthetic construct
  REGION 1..116: note = Anti-HSA-1
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSS

SEQ ID NO: 14 - protein, 8 aa, synthetic construct
  REGION 1..8: note = Anti-HSA-2 CDR1
GFAFRGFG

SEQ ID NO: 15 - protein, 8 aa, synthetic construct
  REGION 1..8: note = Anti-HSA-2 CDR2
INNGGSDT

SEQ ID NO: 16 - protein, 9 aa, synthetic construct
  REGION 1..9: note = Anti-HSA-2 CDR3
AIGGPGASP

SEQ ID NO: 17 - protein, 25 aa, synthetic construct
  REGION 1..25: note = Anti-HSA-2 FR1
QVQLVESGGGVVQPGGSLRLSCAAS

SEQ ID NO: 18 - protein, 17 aa, synthetic construct
  REGION 1..17: note = Anti-HSA-2 FR2
MSWVRQAPGKGLEWVSS

SEQ ID NO: 19 - protein, 11 aa, synthetic construct
  REGION 1..11: note = Anti-HSA-2 FR4
SGQGTQVTVSS

SEQ ID NO: 20 - protein, 116 aa, synthetic construct
  REGION 1..116: note = Anti-HSA-2
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSS

SEQ ID NO: 21 - protein, 6 aa, synthetic construct
  REGION 1..6: note = (GSG)2 Linker
GSGGSG

SEQ ID NO: 22 - protein, 9 aa, synthetic construct
  REGION 1..9: note = (GSG)3 Linker
GSGGSGGSG

SEQ ID NO: 23 - protein, 12 aa, synthetic construct
  REGION 1..12: note = (GSG)4 Linker
GSGGSGGSGGSG

SEQ ID NO: 24 - protein, 4 aa, synthetic construct
  REGION 1..4: note = G3S Linker
GGGS

SEQ ID NO: 25 - protein, 8 aa, synthetic construct
  REGION 1..8: note = (G3S)2 Linker
GGGSGGGS

SEQ ID NO: 26 - protein, 12 aa, synthetic construct
  REGION 1..12: note = (G3S)3 Linker
GGGSGGGSGGGS

SEQ ID NO: 27 - protein, 16 aa, synthetic construct
  REGION 1..16: note = (G3S)4 Linker
GGGSGGGSGGGSGGGS

SEQ ID NO: 28 - protein, 5 aa, synthetic construct
  REGION 1..5: note = G4S Linker
GGGGS

SEQ ID NO: 29 - protein, 10 aa, synthetic construct
  REGION 1..10: note = (G4S)2 Linker
GGGGSGGGGS

SEQ ID NO: 30 - protein, 15 aa, synthetic construct
  REGION 1..15: note = (G4S)3 Linker
GGGGSGGGGSGGGGS

SEQ ID NO: 31 - protein, 20 aa, synthetic construct
  REGION 1..20: note = (G4S)4 Linker
GGGGSGGGGSGGGGSGGGGS

SEQ ID NO: 32 - protein, 4 aa, synthetic construct
  REGION 1..4: note = SGSG Linker
SGSG

SEQ ID NO: 33 - protein, 7 aa, synthetic construct
  REGION 1..7: note = S(GSG)2 Linker
SGSGGSG

SEQ ID NO: 34 - protein, 10 aa, synthetic construct
  REGION 1..10: note = S(GSG)3 Linker
SGSGGSGGSG

SEQ ID NO: 35 - protein, 13 aa, synthetic construct
  REGION 1..13: note = S(GSG)4 Linker
SGSGGSGGSGGSG

SEQ ID NO: 36 - protein, 5 aa, synthetic construct
  REGION 1..5: note = SG3S Linker
SGGGS

SEQ ID NO: 37 - protein, 9 aa, synthetic construct
  REGION 1..9: note = S(G3S)2 Linker
SGGGSGGGS

SEQ ID NO: 38 - protein, 13 aa, synthetic construct
  REGION 1..13: note = S(G3S)3 Linker
SGGGSGGGSGGGS

SEQ ID NO: 39 - protein, 17 aa, synthetic construct
  REGION 1..17: note = S(G3S)4 Linker
SGGGSGGGSGGGSGGGS

SEQ ID NO: 40 - protein, 6 aa, synthetic construct
  REGION 1..6: note = SG4S Linker
SGGGGS

SEQ ID NO: 41 - protein, 11 aa, synthetic construct
  REGION 1..11: note = S(G4S)2 Linker
SGGGGSGGGGS

SEQ ID NO: 42 - protein, 16 aa, synthetic construct
  REGION 1..16: note = S(G4S)3 Linker
SGGGGSGGGGSGGGGS

SEQ ID NO: 43 - protein, 21 aa, synthetic construct
  REGION 1..21: note = S(G4S)4 Linker
SGGGGSGGGGSGGGGSGGGGS

SEQ ID NO: 44 - protein, 5 aa, synthetic construct
  REGION 1..5: note = EAAAK Linker
EAAAK

SEQ ID NO: 45 - protein, 10 aa, synthetic construct
  REGION 1..10: note = (EAAAK)2 Linker
EAAAKEAAAK

SEQ ID NO: 46 - protein, 15 aa, synthetic construct
  REGION 1..15: note = (EAAAK)3 Linker
EAAAKEAAAKEAAAK

SEQ ID NO: 47 - protein, 20 aa, synthetic construct
  REGION 1..20: note = (EAAAK)4 Linker
EAAAKEAAAKEAAAKEAAAK

SEQ ID NO: 48 - protein, 5 aa, synthetic construct
  REGION 1..5: note = PAPAP Linker
PAPAP

SEQ ID NO: 49 - protein, 10 aa, synthetic construct
  REGION 1..10: note = (PAPAP)2 Linker
PAPAPPAPAP

SEQ ID NO: 50 - protein, 15 aa, synthetic construct
  REGION 1..15: note = (PAPAP)3 Linker
PAPAPPAPAPPAPAP

SEQ ID NO: 51 - protein, 20 aa, synthetic construct
  REGION 1..20: note = (PAPAP)4 Linker
PAPAPPAPAPPAPAPPAPAP

SEQ ID NO: 52 - protein, 8 aa, synthetic construct
  REGION 1..8: note = VLVH Linker
IKRTVAAP

SEQ ID NO: 53 - protein, 5 aa, synthetic construct
  REGION 1..5: note = RAKPS Linker
RAKPS

SEQ ID NO: 54 - protein, 10 aa, synthetic construct
  REGION 1..10: note = (RAKPS)2 Linker
RAKPSRAKPS

SEQ ID NO: 55 - protein, 5 aa, synthetic construct
  REGION 1..5: note = ASTKG Linker
ASTKG

SEQ ID NO: 56 - protein, 10 aa, synthetic construct
  REGION 1..10: note = (ASTKG)2 Linker
ASTKGASTKG

SEQ ID NO: 57 - protein, 5 aa, synthetic construct
  REGION 1..5: note = AKTHT Linker
AKTHT

SEQ ID NO: 58 - protein, 10 aa, synthetic construct
  REGION 1..10: note = (AKTHT)2 Linker
AKTHTAKTHT

SEQ ID NO: 59 - protein, 15 aa, synthetic construct
  REGION 1..15: note = ASTKG(G4S)2 Linker
ASTKGGGGGSGGGGS

SEQ ID NO: 60 - protein, 15 aa, synthetic construct
  REGION 1..15: note = AKTHT(G4S)2 Linker
AKTHTGGGGSGGGGS

SEQ ID NO: 61 - protein, 15 aa, synthetic construct
  REGION 1..15: note = RAKPS(G4S)2 Linker
RAKPSGGGGSGGGGS

SEQ ID NO: 62 - protein, 22 aa, synthetic construct
  REGION 1..22: note = hIL-2 fragment (N29 to A50)
NNYKNPKLTRMLTFKFYMPKKA

SEQ ID NO: 63 - protein, 19 aa, synthetic construct
  REGION 1..19: note = IL-15 hinge fragment-containing peptide 1
NNYENPKLTDATLYMPKKA

SEQ ID NO: 64 - protein, 18 aa, synthetic construct
  REGION 1..18: note = IL-15 hinge fragment-containing peptide 2
QSMEIDATTFKFYMPKKA

SEQ ID NO: 65 - protein, 16 aa, synthetic construct
  REGION 1..16: note = IL-15 hinge fragment-containing peptide 3
QSMEIDATKFYMPKKA

SEQ ID NO: 66 - protein, 16 aa, synthetic construct
  REGION 1..16: note = IL-15 hinge fragment-containing peptide 4
QSMEIDATLFYMPKKA

SEQ ID NO: 67 - protein, 15 aa, synthetic construct
  REGION 1..15: note = IL-15 hinge fragment-containing peptide 5
QSMEIDATLYTESDV

SEQ ID NO: 68 - protein, 19 aa, synthetic construct
  REGION 1..19: note = IL-15 hinge fragment-containing peptide 6
NNYHNPKLTDATLYMPKKA

SEQ ID NO: 69 - protein, 18 aa, synthetic construct
  REGION 1..18: note = IL-15 hinge fragment-containing peptide 7
QSMHIDATTFKFYMPKKA

SEQ ID NO: 70 - protein, 16 aa, synthetic construct
  REGION 1..16: note = IL-15 hinge fragment-containing peptide 8
QSMHIDATKFYMPKKA

SEQ ID NO: 71 - protein, 16 aa, synthetic construct
  REGION 1..16: note = IL-15 hinge fragment-containing peptide 9
QSMHIDATLFYMPKKA

SEQ ID NO: 72 - protein, 15 aa, synthetic construct
  REGION 1..15: note = IL-15 hinge fragment-containing peptide 10
QSMHIDATLYTESDV

SEQ ID NO: 73 - protein, 130 aa, synthetic construct
  REGION 1..130: note = IL-2-IL-15 chimera 1 (C125S)
APTSSSTKKTQLQLEHLLLDLQMILNGINNYENPKLTDATLYMPKKATELKHLQCLEEELKPLEEVLNLAQSKNFHLRPRDLISNINVIVLELKGSETTFMCEYADETATIVEFLNRWITFSQSIISTLT

SEQ ID NO: 74 - protein, 129 aa, synthetic construct
  REGION 1..129: note = IL-2-IL-15 chimera 2 (C125S)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 75 - protein, 127 aa, synthetic construct
  REGION 1..127: note = IL-2-IL-15 chimera 3 (C125S)
APTSSSTKKTQLQLEHLLLDLQMILNGIQSMEIDATKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 76 - protein, 127 aa, synthetic construct
  REGION 1..127: note = IL-2-IL-15 chimera 4 (C125S)
APTSSSTKKTQLQLEHLLLDLQMILNGIQSMEIDATLFYMPKKATELKHLQCLEEELKPLEEVLNLAQSKNFHLRPRDLISNINVIVLELKGSETTFMCEYADETATIVEFLNRWITFSQSIISTLT

SEQ ID NO: 77 - protein, 126 aa, synthetic construct
  REGION 1..126: note = IL-2-IL-15 chimera 5 (C125S)
APTSSSTKKTQLQLEHLLLDLQMILNGIQSMEIDATLYTESDVTELKHLQCLEEELKPLEEVLNLAQSKNFHLRPRDLISNINVIVLELKGSETTFMCEYADETATIVEFLNRWITFSQSIISTLT

SEQ ID NO: 78 - protein, 129 aa, synthetic construct
  REGION 1..129: note = IL-2-IL-15 chimera 6 (C125S)
APTSSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 79 - protein, 129 aa, synthetic construct
  REGION 1..129: note = IL-2-IL-15 chimera 7 (E15K, C125S)
APTSSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 80 - protein, 129 aa, synthetic construct
  REGION 1..129: note = IL-2-IL-15 chimera 8 (S87D, C125S)
APTSSSTKKTQLQLEHLLLDLQMILNGIQSMEIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 81 - protein, 129 aa, synthetic construct
  REGION 1..129: note = IL-2-IL-15 chimera 9 (S87E, C125S)
APTSSSTKKTQLQLEHLLLDLQMILNGIQSMEIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 82 - protein, 129 aa, synthetic construct
  REGION 1..129: note = IL-2-IL-15 chimera 10 (E15K, S87D, C125S)
APTSSSTKKTQLQLKHLLLDLQMILNGIQSMEIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 83 - protein, 129 aa, synthetic construct
  REGION 1..129: note = IL-2-IL-15 chimera 11 (E15K, S87E, C125S)
APTSSSTKKTQLQLKHLLLDLQMILNGIQSMEIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 84 - protein, 129 aa, synthetic construct
  REGION 1..129: note = IL-2-IL-15 chimera 12 (E15K, C125S)
APTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 85 - protein, 129 aa, synthetic construct
  REGION 1..129: note = IL-2-IL-15 chimera 13 (S87D, C125S)
APTSSSTKKTQLQLEHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 86 - protein, 129 aa, synthetic construct
  REGION 1..129: note = IL-2-IL-15 chimera 14 (S87E, C125S)
APTSSSTKKTQLQLEHLLLDLQMILNGIQSMHIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 87 - protein, 129 aa, synthetic construct
  REGION 1..129: note = IL-2-IL-15 chimera 15 (E15K, S87D, C125S)
APTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 88 - protein, 129 aa, synthetic construct
  REGION 1..129: note = IL-2-IL-15 chimera 16 (E15K, S87E, C125S)
APTSSSTKKTQLQLKHLLLDLQMILNGIQSMHIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 89 - protein, 130 aa, synthetic construct
  REGION 1..130: note = IL-2-IL-15 chimera 17 (T3A, C125S)
APASSSTKKTQLQLEHLLLDLQMILNGINNYENPKLTDATLYMPKKATELKHLQCLEEELKPLEEVLNLAQSKNFHLRPRDLISNINVIVLELKGSETTFMCEYADETATIVEFLNRWITFSQSIISTLT

SEQ ID NO: 90 - protein, 129 aa, synthetic construct
  REGION 1..129: note = IL-2-IL-15 chimera 18 (T3A, C125S)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 91 - protein, 127 aa, synthetic construct
  REGION 1..127: note = IL-2-IL-15 chimera 19 (T3A, C125S)
APASSSTKKTQLQLEHLLLDLQMILNGIQSMEIDATKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 92 - protein, 127 aa, synthetic construct
  REGION 1..127: note = IL-2-IL-15 chimera 20 (T3A, C125S)
APASSSTKKTQLQLEHLLLDLQMILNGIQSMEIDATLFYMPKKATELKHLQCLEEELKPLEEVLNLAQSKNFHLRPRDLISNINVIVLELKGSETTFMCEYADETATIVEFLNRWITFSQSIISTLT

SEQ ID NO: 93 - protein, 126 aa, synthetic construct
  REGION 1..126: note = IL-2-IL-15 chimera 21 (T3A, C125S)
APASSSTKKTQLQLEHLLLDLQMILNGIQSMEIDATLYTESDVTELKHLQCLEEELKPLEEVLNLAQSKNFHLRPRDLISNINVIVLELKGSETTFMCEYADETATIVEFLNRWITFSQSIISTLT

SEQ ID NO: 94 - protein, 129 aa, synthetic construct
  REGION 1..129: note = IL-2-IL-15 chimera 22 (T3A, C125S)
APASSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 95 - protein, 129 aa, synthetic construct
  REGION 1..129: note = IL-2-IL-15 chimera 23 (T3A, E15K, C125S)
APASSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 96 - protein, 129 aa, synthetic construct
  REGION 1..129: note = IL-2-IL-15 chimera 24 (T3A, S87D, C125S)
APASSSTKKTQLQLEHLLLDLQMILNGIQSMEIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 97 - protein, 129 aa, synthetic construct
  REGION 1..129: note = IL-2-IL-15 chimera 25 (T3A, S87E, C125S)
APASSSTKKTQLQLEHLLLDLQMILNGIQSMEIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 98 - protein, 129 aa, synthetic construct
  REGION 1..129: note = IL-2-IL-15 chimera 26 (T3A, E15K, S87D, C125S)
APASSSTKKTQLQLKHLLLDLQMILNGIQSMEIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 99 - protein, 129 aa, synthetic construct
  REGION 1..129: note = IL-2-IL-15 chimera 27 (T3A, E15K, S87E, C125S)
APASSSTKKTQLQLKHLLLDLQMILNGIQSMEIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 100 - protein, 129 aa, synthetic construct
  REGION 1..129: note = IL-2-IL-15 chimera 28 (T3A, E15K, C125S)
APASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 101 - protein, 129 aa, synthetic construct
  REGION 1..129: note = IL-2-IL-15 chimera 29 (T3A, S87D, C125S)
APASSSTKKTQLQLEHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 102 - protein, 129 aa, synthetic construct
  REGION 1..129: note = IL-2-IL-15 chimera 30 (T3A, S87E, C125S)
APASSSTKKTQLQLEHLLLDLQMILNGIQSMHIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 103 - protein, 129 aa, synthetic construct
  REGION 1..129: note = IL-2-IL-15 chimera 31 (T3A, E15K, S87D, C125S)
APASSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 104 - protein, 129 aa, synthetic construct
  REGION 1..129: note = IL-2-IL-15 chimera 32 (T3A, E15K, S87E, C125S)
APASSSTKKTQLQLKHLLLDLQMILNGIQSMHIDATTFKFYMPKKATELKHLQCLEEELKPLEEVLNLAQSKNFHLRPRDLIENINVIVLELKGSETTFMCEYADETATIVEFLNRWITFSQSIISTLT

SEQ ID NO: 105 - protein, 269 aa, synthetic construct
  REGION 1..269: note = hIL-2 mutein-anti-HSA (C125S) C1
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSGGGGSEVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSS

SEQ ID NO: 106 - protein, 260 aa, synthetic construct
  REGION 1..260: note = Anti-HSA-IL-2 A1 (IL-2-IL15 chimera 7)
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSSGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 107 - protein, 260 aa, synthetic construct
  REGION 1..260: note = Anti-HSA-IL-2 A2 (IL-2-IL15 chimera 12)
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSSGGGGSGGGGSGGGGSAPTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 108 - protein, 260 aa, synthetic construct
  REGION 1..260: note = Anti-HSA-IL-2 A3 (IL-2-IL15 chimera 7)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 109 - protein, 260 aa, synthetic construct
  REGION 1..260: note = Anti-HSA-IL-2 A4 (IL-2-IL15 chimera 12)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSAPTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 110 - protein, 260 aa, synthetic construct
  REGION 1..260: note = Anti-HSA-IL-2 A5 (IL-2-IL15 chimera 15)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSRAKPSGGGGSGGGGSAPTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 111 - protein, 261 aa, synthetic construct
  REGION 1..261: note = Anti-HSA-IL-2 A6 (IL-2-IL15 chimera 15)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSAKTHTGGGGSGGGGSSAPTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 112 - protein, 260 aa, synthetic construct
  REGION 1..260: note = Anti-HSA-IL-2 A7 (IL-2-IL15 chimera 23)
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSSGGGGSGGGGSGGGGSAPASSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 113 - protein, 260 aa, synthetic construct
  REGION 1..260: note = Anti-HSA-IL-2 A8 (IL-2-IL15 chimera 28)
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSSGGGGSGGGGSGGGGSAPASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 114 - protein, 260 aa, synthetic construct
  REGION 1..260: note = Anti-HSA-IL-2 A9 (IL-2-IL15 chimera 23)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSAPASSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 115 - protein, 260 aa, synthetic construct
  REGION 1..260: note = Anti-HSA-IL-2 A10 (IL-2-IL15 chimera 28)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSAPASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 116 - protein, 260 aa, synthetic construct
  REGION 1..260: note = Anti-HSA-IL-2 A11 (IL-2-IL15 chimera 31)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSRAKPSGGGGSGGGGSAPASSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 117 - protein, 261 aa, synthetic construct
  REGION 1..261: note = Anti-HSA-IL-2 A12 (IL-2-IL15 chimera 31)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSAKTHTGGGGSGGGGSSAPASSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 118 - protein, 129 aa, synthetic construct
  REGION 1..129: note = IL-2-IL-15 chimera 33 (L18C, Q126E)
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 119 - protein, 129 aa, synthetic construct
  REGION 1..129: note = IL-2-IL-15 chimera 34 (T3A, L18C, Q126E)
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 120 - protein, 260 aa, synthetic construct
  REGION 1..260: note = Anti-HSA-IL-2 A13 (IL-2-IL15 chimera 33)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSAPTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 121 - protein, 260 aa, synthetic construct
  REGION 1..260: note = Anti-HSA-IL-2 A14 (IL-2-IL15 chimera 34)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSAPASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 122 - protein, 260 aa, synthetic construct
  REGION 1..260: note = Anti-HSA-IL-2 A15 (IL-2-IL15 chimera 12)
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSSASTKGGGGGSGGGGSAPTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 123 - protein, 260 aa, synthetic construct
  REGION 1..260: note = Anti-HSA-IL-2 A16 (IL-2-IL15 chimera 28)
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSSASTKGGGGGSGGGGSAPASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 124 - protein, 260 aa, synthetic construct
  REGION 1..260: note = Anti-HSA-IL-2 A17 (IL-2-IL15 chimera 12)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSASTKGGGGGSGGGGSAPTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 125 - protein, 260 aa, synthetic construct
  REGION 1..260: note = Anti-HSA-IL-2 A18 (IL-2-IL15 chimera 28)
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSASTKGGGGGSGGGGSAPASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT